(19) United States
(12) Patent Application Publication
(10) Pub. No.: US 2013/0029874 A1
(43) Pub. Date: Jan. 31, 2013

(54) MICRORNA MARKERS FOR RECURRENCE OF COLORECTAL CANCER

(71) Applicants: Elizabeth Mambo, Austin, TX (US); Timothy S. Davison, Austin, TX (US); Paul A. LeBourgeois, Austin, TX (US)

(72) Inventors: Elizabeth Mambo, Austin, TX (US); Timothy S. Davison, Austin, TX (US); Paul A. LeBourgeois, Austin, TX (US)

(21) Appl. No.: 13/646,509

(22) Filed: Oct. 5, 2012

Related U.S. Application Data
(63) Continuation of application No. 12/398,852, filed on Mar. 5, 2009, now abandoned.

(51) Int. Cl. C12Q 1/68; C40B 40/06; C40B 30/04

(52) U.S. Cl. 506/9; 435/6.14; 435/6.12; 435/6.11; 506/16

(57) ABSTRACT

The present invention concerns methods and compositions for identifying a miRNA profile for a particular condition, such as colorectal cancer, and using the profile in the diagnosis and/or prognosis of a patient for a condition, such as colorectal cancer and colorectal cancer recurrence or response to therapy.

This application claims priority to U.S. Provisional Patent Application Ser. No. 61/034,422 filed on Mar. 6, 2008, which is incorporated herein by reference in its entirety.

### BACKGROUND OF THE INVENTION

I. Field of the Invention

The present invention relates generally to the fields of molecular biology and oncology. More particularly, it concerns methods and compositions involving microRNA (miRNAs) molecules and cancer diagnosis and/or prognosis. Certain aspects of the invention include applications for miRNAs in diagnosis and prognosis of colorectal cancer.

II. Background

Cancer remains a serious public health problem in the United States and other developed countries. Currently, one in four deaths in the United States is due to cancer (Jemal et al., 2007). Colorectal cancer refers to cancerous growths that occur in the colon and rectum. In the United States, colorectal cancer is the third most common type of cancer and also the third leading cause of cancer mortality in both men and women (Jemal et al., 2007). Only a handful of treatments are available for specific types of cancer, and these provide no guarantee of success. To be most effective, cancer treatment requires not only early detection and treatment or removal of the malignancy, but a reliable assessment of the severity of the malignancy and a prediction of the likelihood of cancer recurrence.

At present, no surrogate markers are validated for predicting colon cancer recurrence at the time of surgery. CEA is an FDA-approved marker only for surveillance of recurrent colorectal cancer following surgery. CEA cannot predict recurrence as it is not colon specific, it can be elevated in a variety of other carcinomas (e.g., gastric, pancreatic, lung, breast, and ovary), and it can be elevated in benign conditions (e.g., hepatic cirrhosis, inflammatory bowel disease, chronic lung disease, pancreatitis and cigarette-smoking patients). Of note, CEA is not detected in all cases of colorectal cancer, and the presence and/or absence of the antigen at the time of surgery is not predictive of recurrence.

A need exists for additional colon cancer markers that are predictive of recurrence in early clinical stage disease.

### SUMMARY OF THE INVENTION

The present invention provides additional methods for diagnosis and prognosis of colorectal cancer by, in certain aspects, identifying miRNAs that are differentially expressed or mis-regulated in various states of diseased, normal, cancerous, and/or abnormal tissues, including but not limited to normal colon and colorectal cancer. Further, the invention describes a method for diagnosing colorectal cancer that is based on determining levels (increased or decreased) of selected miRNAs in patient-derived samples. In certain aspects, RNA may or may not be isolated from a sample. In other aspects, sample RNA will be coupled to a label and/or a probe. In a further aspect, data from RNA assessment will be transformed into a score or index indicative of expression levels.

The term “miRNA” or “miR” is used according to its ordinary and plain meaning and refers to a microRNA molecule found in eukaryotes that is involved in RNA-based gene regulation. See, e.g., Carrington et al., 2003, which is hereby incorporated by reference. The term will be used to refer to the single-stranded RNA molecule processed from a precursor. Names of miRNAs and their sequences related to the present invention are provided herein.

miR sequences can be used to evaluate colorectal tissue for the possibility of a hyperproliferative condition of the colon that is characterized by the presence of uncontrolled or hyperactive cell division that results in disease or a pathological condition. Hyperproliferative conditions include colorectal cancer, an invasive and/or metastatic hyperproliferative condition, and precancers.

In certain aspects, an miRNA that is differentially expressed between colorectal cancer tissue and normal adjacent tissue is used to assess a patient having or suspected of having colorectal cancer, e.g. diagnosing and/or prognosing the patient's condition. An miRNA used to diagnose or prognose colorectal cancer can include one or more of hsa-miR-215, hsa-miR-451, m hsa-miR-422a, hsa-miR-422b, hsa-miR-133b, hsa-miR-133a, hsa-miR-195, hsa-miR-194, hsa-miR43, hsa-miR-30c, hsa-miR-192, hsa-miR-497, hsa-miR-1, hsa-miR-375, hsa-miR-145, hsa-miR-150, hsa-miR-30b, hsa-cand342, hsa-miR-183, hsa-miR-182, hsa-miR30, hsa-miR-224, hsa-miR-31, hsa-miR-143, hsa-miR-30a-5p, hsa-cand26, hsa-miR-10b, and hsa-miR-30e-5p.

In certain aspects one or more miRNA selected from hsa-miR-451, hsa-miR-422a, hsa-miR-195, hsa-miR-194, hsa-miR43, hsa-miR-192, hsa-miR-497, hsa-miR-1, hsa-miR-375, hsa-miR-150, hsa-miR-30b, hsa-cand342, hsa-miR30, hsa-miR-224, hsa-miR-30a-5p, hsa-cand26, hsa-miR-10b, and hsa-miR-30e-5p, or combinations thereof, can be used in the diagnosis and prognosis of colorectal cancer.

In still a further aspect one or more miRNA can be used to assess the likelihood of colorectal cancer recurrence by evaluating the expression of one or more miRNA selected from hsa-miR-1, hsa-miR-20a, hsa-miR-194, hsa-miR-203, hsa-miR-26b, hsa-miR-15a, hsa-miR-133b, hsa-miR-107, hsa-miR-141, hsa-miR-155, hsa-miR-20b, hsa-miR-195, hsa-miR-106a, hsa-miR-29b, hsa-miR-223, hsa-miR-17-5p, hsa-miR-103, hsa-miR-660, hsa-let-7g, hsa-miR-15b, hsa-miR-23a, hsa-miR-182, hsa-miR-29a, hsa-miR-98, hsa-miR-16, hsa-miR43, hsa-miR-106b, hsa-miR-30b, hsa-miR-27a, hsa-miR-19b, hsa-miR-27b, hsa-miR-342, hsa-miR-146a, hsa-miR-361, hsa-miR-93, hsa-miR257, hsa-miR-130a, hsa-miR-152, hsa-miR-335, hsa-miR-143, hsa-miR-28, hsa-miR-30e-5p, hsa-miR-25, hsa-miR-146b, hsa-cand144, hsa-miR-95, hsa-miR-218, hsa-miR-128a, hsa-let-71, hsa-miR-34a, hsa-miR-130b, hsa-miR-21, hsa-miR-30a-5p, hsa-miR-30a-3p, hsa-miR-652, hsa-miR-625, hsa-miR-191, hsa-miR-17-3p, hsa-miR-222, and/or hsa-miR-594. In certain aspects the assessment is independent of the stage of cancer being assessed.

In still yet another aspect, a patient with stage II colorectal cancer can be assessed by evaluating the expression level of one or more miRNA selected from hsa-miR-20a, hsa-miR-196b, hsa-miR-196a, hsa-miR-155, hsa-miR-194, hsa-miR-7, hsa-miR-98, hsa-miR-106a, hsa-miR-182, hsa-miR-26b, hsa-miR-17-5p, hsa-miR-15a, hsa-miR-146a, hsa-miR-20b, hsa-miR-148a, hsa-miR-106b, hsa-miR-15b, hsa-miR-660, hsa-miR-29b, hsa-miR-335, hsa-miR-93, hsa-miR-107, hsa-let-7g, hsa-miR-19b, hsa-miR-25, hsa-miR-29a, hsa-miR-152, hsa-miR-103, hsa-miR-146b, hsa-miR-128a, hsa-let-7f, hsa-miR-16, hsa-miR-34a, hsa-miR-218, hsa-miR-222, hsa-miR-28, hsa-miR-221, hsa-miR-652, hsa-miR-181d, hsa-let-71, hsa-miR-191, and/or hsa-miR-185.

In still yet another aspect, a patient with stage II colorectal cancer can be assessed by evaluating the expression level of one or more miRNA selected from the group consisting of hsa-miR-15b, hsa-miR-20b, hsa-miR-93, hsa-let-7f, hsa-miR-20a, hsa-miR-19b, hsa-miR-103, hsa-let-7g, hsa-miR-107, hsa-miR-25, hsa-miR-16, hsa-miR-128, hsa-miR-28-5p, hsa-miR-26b, hsa-miR-29a, hsa-miR-221, hsa-miR-29b-1*, hsa-miR-185, hsa-miR-34a, hsa-miR-148a miR-146a miR-155 miR-146b miR-15a let-71 miR-191, hsa-miR-501-5p, hsa-miR-632, hsa-miR-500, hsa-let-7c*, hsa-miR-125b-2*, hsa-miR-892b, hsa-miR-139-3p, hsa-miR-596, hsa-miR-135b*, hsa-miR-302c*, and/or hsa-miR-675.

In certain aspects patients with stage III colorectal cancer can be assessed for recurrence and/or response to therapy by evaluating expression levels of one or more of miRNA hsa-miR-133a, hsa-miR-133b, hsa-miR-205, and/or hsa-cand173.

Corresponding miRNA sequences that can be used in the context of the invention include, but are not limited to, all or a portion of those sequences in the sequence listing provided herein, as well as the miRNA precursor sequence, or complement of one or more of these miRNAs.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more miRNA. Consequently, in some embodiments, methods include a step of generating a miRNA profile for a sample. The term “miRNA profile” refers to data regarding the expression pattern of miRNAs in the sample (e.g., one or more miRNA from Table 3, 4, 5, 6, 7, 10 and/or 11). It is contemplated that the miRNA profile can be obtained using a set of miRNAs, using for example nucleic acid amplification or hybridization techniques well known to one of ordinary skill in the art. In certain embodiments, expression of one or more miRNA from Table 3, 4, 5, 6, 7, 10 and/or 11 is evaluated. In a further aspect, a set or subset of miRNAs may include, or specifically exclude, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 70, 75, 80, 90, 100 or more miRNA described herein, including all values and ranges there between (e.g., those listed in Tables 3, 4, 5, 6, 7, 10 and 11).

In some embodiments of the invention, an miRNA profile is generated by steps that include one or more of: (a) labeling miRNA in the sample; (b) hybridizing miRNA to a number of probes, or amplifying a number of miRNA, and/or (c) determining miRNA hybridization to the probes or detecting miRNA amplification products, wherein a miRNA expression levels are determined or evaluated. See U.S. Provisional Patent Applications 60/575,743 and 60/649,584, and U.S. patent application Ser. Nos. 11/141,707 and 11/855,792, all of which are hereby incorporated by reference.

Methods of the invention include determining a diagnosis or prognosis for a patient based on miRNA expression or expression levels. In certain embodiments, the elevation or reduction in the level of expression of a particular miRNA or set of miRNA in a cell is correlated with a disease state as compared to the expression level of that miRNA or set of miRNA in a normal cell or a reference sample or digital reference or a scale. In certain embodiments, if the assessment of a sample fulfills certain criteria, the patient is diagnosed with a hyperproliferative or cancerous disorder or condition. In certain embodiments, a scale is used to measure a sign, symptom, or symptom cluster of a disorder, and the disorder is diagnosed on the basis of the measurement using that scale. In certain embodiments, a “score” on a scale is used to diagnose or assess a sign, symptom, or symptom cluster of a disorder. In certain embodiments, a “score” can measure at least one of the frequency, intensity, or severity of a sign, symptom, or symptom cluster (such as the expression level of one or more miRNA) of a disorder. A scale will typically have a threshold value and the relationship of a score derived from assessing a sample will determine the prognosis or diagnosis of a patient. This allows for diagnostic methods to be carried out when the expression level of a miRNA is measured in a biological sample being assessed. In certain aspects the miRNA expression level is compared to the expression level of a normal cell or a reference sample or a digital reference. It is specifically contemplated that miRNA profiles for patients, particularly those suspected of having or at risk of developing (e.g., a recurrence of cancer) a particular disease or condition such as colorectal cancer, can be generated by evaluating any miR or set of miRs discussed in this application. The miRNA profile that is generated from the patient will be one that provides information regarding the particular disease or condition. In certain aspects, a party evaluating miR expression may prepare a recommendation, report and/or summary conveying processed or raw data to a diagnosing physician. In certain aspects, a miRNA profile can be used in conjunction with other diagnostic tests.

In a still further aspect the methods of the invention can be used to assess the likelihood of colorectal cancer recurrence in a patient comprising determining the expression levels of miRNA in a sample comprising colorectal cancer cells and determining a prognosis for colorectal cancer recurrence based on the miRNA expression levels. A recurrence is typically a second instance of cancer in a patient. In certain aspects the second occurrence is in the same tissue or in a second tissue (e.g. non-rectal or non-colon tissue). In still a further aspect the second occurrence is in the same location, proximal to, or distant to the first occurrence. In yet a further aspect the first instance of cancer is Stage I, II, III, or IV cancer.

Embodiments of the invention include methods for diagnosing, assessing a condition, and/or prognosing a disease, such as cancer recurrence, in a patient comprising evaluating or determining the expression or expression levels of one or more miRNAs in a sample from the patient. The difference in the expression in the sample from the patient and a reference, such as expression in a normal or non-pathologic sample, is indicative of a pathologic, disease, or cancerous condition. An miRNA, amplification product, or probe set can comprise a segment of or be complementary to a corresponding miRNA including all or part of miRNA sequence described herein. In certain aspects of the invention, a segment can comprise 2, 3, 4, 5, 6, 7, 8, 9, 10, 11, 12 or more nucleotide sequences of a miRNA. Other amplification or hybridization sequences may also be included for normalization purposes. The use of an miRNA quantification assay as a clinically relevant diagnostic tool can be enhanced by using an appropriate normalization control. The methods of normalization correct for sample-to-sample variability by comparing a target measurement in a sample to one or more internal controls. Normalization of miRNA quantification assays reduces systematic (non-biological) and non-systematic differences between samples, and can enhance the accurate measurement of differential miRNA expression, for example. The accurate measurement of biologically hardwired differential expression between two groups of samples is the goal of many miRNA qRT-PCR assays. Yet, miRNA levels in qRT-PCR reactions can vary from one sample to the next for reasons that may be technical or biological. Technical reasons may include variations in tissue procurement or storage, inconsistencies in RNA extraction or quantification, or differences in the efficiency of the reverse transcription and/or PCR steps. Biological reasons may include sample-to-sample heterogeneity in cellular populations, differences in bulk transcriptional activity, or alterations in specific miRNA expression that is linked to an aberrant biological program (e.g., a disease state). Given the multiplicity of sources that can contribute to differences in miRNA quantification, results from qRT-PCR assays can be normalized against a relevant endogenous target or targets to minimize controllable variation, and permit definitive interpretations of nominal differences in miRNA expression.

With no intent of limiting the invention to any particular theory, most cancers are initially recognized either because signs or symptoms appear, or are identified through screening. Neither of these leads to a definitive diagnosis, which usually requires the opinion of a pathologist. Typically, people with suspected cancer are investigated with various medical tests. These commonly include blood tests, X-rays, CT scans and endoscopy.

A cancer may be suspected for a variety of reasons, but diagnosis of most malignancies is typically confirmed by histological examination of the cancerous cells by a pathologist. Tissue can be obtained from a biopsy or surgery. Many biopsies (such as those of the skin, breast or liver) can be done in a doctor's office. Biopsies of other organs are performed under anesthesia and may require surgery in an operating room.

The tissue diagnosis indicates the type of cell that is proliferating, its histological grade and other features of the tumor. Together, this information is useful to evaluate the prognosis of this patient and choose the best treatment. Cytogenetics and immunohistochemistry may provide information about future behavior of the cancer (prognosis) and best treatment.

A physician may choose to treat a cancer by surgery, chemotherapy, radiation therapy, immunotherapy, monoclonal antibody therapy and/or other methods. The choice of therapy depends upon the location and grade of the tumor and the stage of the disease, as well as the general state of the patient.

Because “cancer” refers to a class of diseases, it is unlikely that there will be a single treatment and aspects of the invention can be used to determine which treatment will be most effective or most harmful and provide a guide for the physician in evaluating, assessing and formulating a treatment strategy for a patient.

A sample may be taken from a patient having or suspected of having a disease or pathological condition. In certain aspects, the sample can be, but is not limited to tissue (e.g., biopsy, particularly fine needle biopsy), sputum, lavage fluid, blood, serum, plasma, lymph node or other tissue or fluid that may contain a colon cancer cell. The sample can be fresh, frozen, fixed (e.g., formalin fixed), or embedded (e.g., paraffin embedded). In a particular aspect, the sample can be a colon or rectal sample.

Methods of the invention can be used to diagnose or assess a pathological condition. In certain aspects the condition is a cancerous condition, such as colorectal, colon, or rectal cancer.

Certain embodiments of the invention include determining expression of one or more miRNA by using an amplification assay or a hybridization assay, a variety of which are well known to one of ordinary skill in the art. In certain aspects, an amplification assay can be a quantitative amplification assay, such as quantitative RT-PCR or the like. In still further aspects, a hybridization assay can include array hybridization assays or solution hybridization assays.

The methods can further comprise or exclude one or more of steps including: (a) obtaining a sample from the patient, (b) isolating or obtaining nucleic acids from the sample, (c) reverse transcribing nucleic acids from the sample, (d) labeling the nucleic acids isolated from the sample or an amplification product thereof, (e) hybridizing the labeled nucleic acids to one or more probes or detecting the amplified nucleic acids, (f) analyzing and normalizing data by statistical methods, and/or (g) creating and/or supplying a report of the analysis. Nucleic acids of the invention may include one or more nucleic acid comprising at least one segment having a sequence or complementary sequence of one or more miRNA described herein. In certain aspects, the nucleic acids identify one or more miRNA described herein. Nucleic acids of the invention may be coupled to a support. Such supports are well known to those of ordinary skill in the art and include, but are not limited to glass, plastic, metal, or latex. In particular aspects of the invention, the support can be planar or in the form of a bead or other geometric shapes or configurations.

Aspects of the invention can be used to diagnose or assess a patient's condition. For example, the methods can be used to screen for a pathological condition, assess prognosis of a pathological condition, stage a pathological condition, or assess response of a pathological condition to therapy. In certain aspects it is determined if a patient has colorectal cancer or the recurrence of colorectal cancer. In a further aspect, determining prognosis includes, but is not limited to estimating the likelihood of cancer recurrence.

The present invention also concerns kits containing compositions of the invention or compositions to implement methods of the invention. In some embodiments, kits can be used to evaluate one or more miRNA molecules. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more miRNA probes, synthetic miRNA molecules or miRNA inhibitors, or any range and combination derivable therein. In some embodiments, there are kits for evaluating miRNA activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more, including all values and ranges there between.

Kits for using miRNA probes, synthetic miRNAs, nonsynthetic, and/or miRNA inhibitors of the invention for therapeutic, prognostic, or diagnostic applications are included as part of the invention. Specifically contemplated are any such molecules corresponding to any miRNA diagnostic of colorectal disease, such as those discussed herein.

It is contemplated that any method or composition described herein can be implemented with respect to any other method or composition described herein and that different embodiments may be combined. It is specifically contemplated that any methods and compositions discussed herein with respect to miRNA molecules or miRNA may be implemented with respect to synthetic miRNAs to the extent the synthetic miRNA is exposed to the proper conditions to allow it to become a mature miRNA under physiological circumstances. The claims originally filed are contemplated to cover claims that are multiply dependent on any filed claim or combination of filed claims.

Any embodiment of the invention involving specific miRNAs by name is contemplated also to cover embodiments involving miRNAs whose sequences are at least 80, 81, 82, 83, 84, 85, 86, 87, 88, 89, 90, 91, 92, 93, 94, 95, 96, 97, 98, 99% identical to the mature sequence of the specified miRNA. In other aspects, miRNA of the invention may include additional nucleotides at the 5′, 3′, or both 5′ and 3′ ends of at least, at most or about 1, 2, 3, 4, 5, 6, 7, 8, 9, 10 or more nucleotides.

Embodiments of the invention include kits for analysis of a pathological sample by assessing miRNA profile for a sample comprising, in suitable container means, one or more miRNA probes and/or amplification primers, wherein the miRNA probes detect or primer amplify one or more miRNA described herein. The kit can further comprise reagents for labeling miRNA in the sample. The kit may also include the labeling reagents include at least one amine-modified nucleotide, poly(A) polymerase, and poly(A) polymerase buffer. Labeling reagents can include an amine-reactive dye.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Any embodiment discussed with respect to a particular colorectal disorder can be applied or implemented with respect to a different colorectal disorder. Furthermore, compositions and kits of the invention can be used to achieve methods of the invention.

Throughout this application, the term “about” may be used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 qRT-PCR quantification of four miRNAs in tumors from colorectal cancer patients who had cancer recurrence or who had no recurrence and in normal colon tissue samples. (SII R, patients with stage II cancer and cancer recurrence, n=5; SII NR, patients with stage II cancer and no cancer recurrence, n=5; SIII NR, patients with stage III cancer and no cancer recurrence, n=4; SI NR, patients with stage I cancer and no cancer recurrence, n=1; NCo, normal colon tissue, n=5).

FIG. 2 qRT-PCR quantification of miRNA combinations in tumors from colorectal cancer patients who had cancer recurrence or who had no recurrence. (SII R, patients with stage II cancer and cancer recurrence, n=5, with 3 duplicate samples=8 data points; SII NR, patients with stage II cancer and no cancer recurrence, n=5). ΔΔCT; sum of ΔCT for the indicated miRNAs, where ACT is defined as Cₜ of miRNA of interest —Cₜ of reference miRNA. The reference miRNA used in this example is miRNA-638.

### DETAILED DESCRIPTION OF THE INVENTION

The present invention is directed to compositions and methods relating to preparation and characterization of miRNAs, as well as use of miRNAs for prognostic and/or diagnostic applications, particularly those methods and compositions related to assessing and/or identifying colorectal disease.

microRNAs (miRNAs) are short RNA molecules (17-24 nucleotides in length) that arise from longer precursors, which are transcribed from non-protein-encoding genes. See review of Carrington and Ambros (2003). Recent studies have shown that expression levels of numerous miRNAs are associated specifically with various cancers (reviewed in Esquela-Kerscher and Slack, 2006; Calin and Croce, 2006). miRNAs have also been implicated in regulating cell growth, cell proliferation, and cell and tissue differentiation—cellular processes that are associated with the development of cancer. Studies related to colorectal cancer include:

Akao et al. have evaluated the roles of let-7 (Akao et al., 2006) and miRs-143 and -145 (Akao et al., 2007) in colorectal cancer.

Cummins et al. (2006) describe miRNAs that are differentially expressed in colon cancer and some that are associated with clinical outcome, but do not address miRNAs and colorectal cancer recurrence.

Michael et al. (2003) describe miRNAs that are down-regulated in colorectal adenocarcinomas as compared to matched normal tissues, in particular miR-143 and miR-145, but do not address miRNAs and colorectal cancer recurrence.

Bandres et al. (2006) describe miRNA in paired colorectal tumor and normal adjacent tissue and reported the differential expression of miR-31 in Stage II and Stage IV colorectal cancer cells.

Xi et al. (2006a, 2006b), evaluated the prognostic value of several miRNAs in colorectal cancer and showed that higher expression of miR-200c was associated with shorter survival time. However, the studies did not address association of miRNA expression with colorectal cancer recurrence.

More recently, Schetter et al., (2008) reported that a high expression of miR-21 is associated with a poor survival and poor therapeutic response in colorectal cancer patients. The study also reported that high levels of miR-21 were associated with disease relapse in stage III patients.

Lanza et al. (2007) describe miRNA expression profiles in microsatellite-unstable colorectal cancers.

The present invention advances the current art for colorectal cancer diagnosis and patient prognosis by describing the use of novel miRNA markers for colorectal cancer diagnosis and for the prediction of colorectal cancer recurrence.

### I. COLORECTAL CANCER

Colorectal cancer, also called colon cancer or bowel cancer, includes cancerous growths in the colon, rectum and appendix. It is the third most common form of cancer and the second leading cause of cancer-related death in the Western world. Many colorectal cancers are thought to arise from adenomatous polyps in the colon. These mushroom-like growths are usually benign, but some may develop into cancer over time. The majority of the time, the diagnosis of localized colon cancer is through colonoscopy.

Colorectal cancer can take many years to develop and early detection of colorectal cancer greatly improves the chances of a cure. Therefore, screening for the disease is recommended in individuals who are at increased risk. There are several different tests available for this purpose. These tests include one or more of the following:

Digital rectal exam (DRE): The doctor inserts a lubricated, gloved finger into the rectum to feel for abnormal areas. It only detects tumors large enough to be felt in the distal part of the rectum but is useful as an initial screening test.

Fecal occult blood test (FOBT): a test for blood in the stool.

Sigmoidoscopy: A lighted probe (sigmoidoscope) is inserted into the rectum and lower colon to check for polyps and other abnormalities.

Colonoscopy: A lighted probe called a colonoscope is inserted into the rectum and the entire colon to look for polyps and other abnormalities that may be caused by cancer. A colonoscopy has the advantage that if polyps are found during the procedure they can be immediately removed. Tissue can also be taken for biopsy. In the United States, colonoscopy or FOBT plus sigmoidoscopy are the preferred screening options.

Other methods of screening include:

Double contrast barium enema (DCBE): First, an overnight preparation is taken to cleanse the colon. An enema containing barium sulfate is administered, then air is insufflated into the colon, distending it. The result is a thin layer of barium over the inner lining of the colon which is visible on X-ray films. A cancer or a precancerous polyp can be detected this way. This technique can miss the (less common) flat polyp. Virtual colonoscopy replaces X-ray films in the double contrast barium enema (above) with a special computed tomography scan and requires special workstation software in order for the radiologist to interpret. This technique is approaching colonoscopy in sensitivity for polyps. However, any polyps found must still be removed by standard colonoscopy.

Standard computed axial tomography is an x-ray method that can be used to determine the degree of spread of cancer, but is not sensitive enough to use for screening. Some cancers are found in CAT scans performed for other reasons. Blood tests: Measurement of the patient's blood for elevated levels of certain proteins can give an indication of tumor load. In particular, high levels of carcinoembryonic antigen (CEA) in the blood can indicate metastasis of adenocarcinoma. These tests are frequently false positive or false negative, and are not recommended for screening, but can be useful to assess disease recurrence in patients who are CEA positive. Currently, no methods are available for predicting and/or monitoring colorectal cancer recurrence in patients who are CEA negative.

Genetic counseling and genetic testing for families who may have a hereditary form of colon cancer, such as hereditary nonpolyposis colorectal cancer (HNPCC) or familial adenomatous polyposis (FAP).

Positron emission tomography (PET) is a 3-dimensional scanning technology where a radioactive sugar is injected into the patient, the sugar collects in tissues with high metabolic activity, and an image is formed by measuring the emission of radiation from the sugar. Because cancer cells often have very high metabolic rate, this can be used to differentiate benign and malignant tumors. PET is not used for screening and does not (yet) have a place in routine workup of colorectal cancer cases. Whole-Body PET imaging is the most accurate diagnostic test for detection of recurrent colorectal cancer, and is a cost-effective way to differentiate resectable from non-resectable disease. A PET scan is indicated whenever a major management decision depends upon accurate evaluation of tumor presence and extent.

Stool DNA testing is an emerging technology in screening for colorectal cancer. Pre-malignant adenomas and cancers shed DNA markers from their cells which are not degraded during the digestive process and remain stable in the stool. Capture, followed by Polymerase Chain Reaction amplifies the DNA to detectable levels for assay. Clinical studies have shown a cancer detection sensitivity of 71%-91%.

In the United States, the American Joint Committee on Cancer (AJCC) provides criteria for staging based on the TNM staging system (Greene et al., 2002; Sobin and Wittekind, 2002). The TNM system was developed by the International Union Against Cancer (UICC) and the American Joint Committee on Cancer (AJCC) and attempts to provide a uniform stratification of patients that allows for the comparison of patients in clinical studies and for determining optimal treatment and survival rates. Common elements considered in the TNM and in most other staging systems include location of the primary tumor, tumor size and number of tumors (T1 through T4), lymph node involvement (spread of cancer into lymph nodes, N0 or N1), cell type and tumor grade (how closely the cancer cells resemble normal tissue), and presence or absence of cancer metastasis (M0 or M1). TNM criteria are different for each anatomic cancer site. Once a patient's T, N, and M categories have been determined, usually after surgery, this information is combined with clinical information requisite to the specific cancer type to determine the clinical stage, which is expressed in Roman numerals from stage I (the least advanced stage) to stage IV (the most advanced stage). Cancer staging criteria are modified and updated over time, as scientists learn more about individual cancer types and identify which aspects of the system represent accurate predictors for disease recurrence and patient survival. Often information obtained from clinical trials necessitates a further subdivision of a clinical stage, and these are designated with a Roman numeral and an alpha character (e.g., Stage IIa, IIb, etc.).

Cancer “recurrence”, in pathology nomenclature, refers to cancer re-growth at the site of the primary tumor. For many cancers, such recurrence results from incomplete surgical removal or from micrometastatic lesions in neighboring blood or lymphatic vessels outside of the surgical field. Conversely, “metastasis” refers to a cancer growth distant from the site of the primary tumor. Metastasis of a cancer is believed to result from vascular and/or lymphatic permeation and spread of tumor cells from the site of the primary tumor prior to surgical removal. The prevailing clinical nomenclature used for cancer statistics is somewhat confusing in that patients who experience a second episode of a treated cancer are referred to as having undergone a “recurrence”, whereas these lesions are usually temporally remote metastases at sites distant from the primary cancer. This clinical terminology will be used herein, i.e., the term “recurrence” denotes these late-arising metastatic lesions, unless specific pathologic nomenclature is needed to separate the two forms of clinical recurrence.

For many cancers, lymph node involvement, or metastasis to lymph nodes, has become recognized as a strong predictor of disease recurrence and patient survival. However, 25-35% of cancer patients with no apparent lymph node metastasis, i.e., patients who are “lymph node-negative” for cancer, will experience a recurrence of their cancer. Some believe that cancer recurrence in lymph node-negative patients is due to the presence in the lymph node(s) of occult (i.e., not readily apparent, or hidden) and/or micrometastatic (<2 mm) deposits that are not detected by the present state of the art examination. An alternative hypothesis is that once the requisite changes in the genetics of the primary cancer have been achieved, the biologic potential of that primary cancer has a higher likelihood to result in metastasis than a primary cancer without similar mutations.

The treatment and outlook for colorectal cancer patients, given the current state of the art, depends on the stage of the cancer. The present clinical grouping for colon cancer is shown in Table 1. The two forms of cancer recurrence described above may occur in these patients. Using pathologic nomenclature, a true cancer recurrence is a local recurrence at the excision bed of the primary cancer. This is usually due to incomplete surgical removal of the cancer when the peritoneal surface has been penetrated (T4) and is most often seen in rectal cancer due to the technical difficulty involved in surgery at that site. The majority of clinical recurrences are the appearance of distant metastatic lesions after initial therapy. The risk of clinical recurrence rises with the T class (size and penetration of the primary cancer) and the N class (status of regional lymph node involvement), as well as the histologic type and grade, vascular invasion, and serum carcinoembryonic antigen (CEA) levels. The overall 5-year survival rate in colorectal cancer is 90% in localized cancers (stages 0 and 1) and falls by increasing stage to 9% for distant metastasis (stage IV). Recurrence rates range from 20% in stage I patients, to 40% in stage 11 patients, and up to 60% in stage III patients (Baddi and Benson, 2005; Haller et al., 2005; Meyerhardt et al., 2003; Meyerhardt and Mayer, 2003; Sargent et al., 2005).

TABLE 1
Current AJCC clinical stage groups for colon cancer based on
tumor characteristics (T), lymph node involvement (N), and
cancer metastasis (M). (Greene et al, 2002)

| Clinical | | | |
|---|---|---|---|
| Stage | T | N | M |
| 0 | Tis | N0 | M0 |
| I | T1-2 | N0 | M0 |
| IIA | T3 | N0 | M0 |
| IIB | T4 | N0 | M0 |
| IIIA | T1-T2 | N1 | M0 |
| IIIB | T3-T4 | N1 | M0 |
| IIIC | Any T | N2 | M0 |
| IV | Any T | Any N | M1 |

The main treatment for colorectal cancer is surgical removal of the tumor and nearby lymph nodes, which often results in a cure for approximately 50% of the patients (Chmielarz et al., 2001). Adjuvant therapy (treatment given in addition to the primary therapy) may include chemotherapy and radiation therapy and is typically administered only to stage III and IV patients to reduce the risk of future cancer recurrence. However, cancer recurrence following surgery, even in very early clinical stages, remains a major problem and is often the ultimate cause of death (Meyerhardt and Mayer, 2005). The challenge remains to identify patients having a high risk of colon cancer recurrence at a time when medical intervention will benefit most.

### II. EVALUATION OF MIRNA LEVELS

It is contemplated that a number of assays could be employed to analyze miRNAs, their activities, and their effects. Such assays include, but are not limited to, array hybridization, solution hybridization, nucleic acid amplification, polymerase chain reaction, quantitative PCR, RT-PCR, in situ hybridization, Northern hybridization, hybridization protection assay (HPA) (GenProbe), branched DNA (bDNA) assay (Chiron), rolling circle amplification (RCA), single molecule hybridization detection (US Genomics), Invader assay (ThirdWave Technologies), and/or Oligo Ligation Assay (OLA), hybridization, and array analysis.

U.S. patent application Ser. Nos. 11/141,707, filed May 31, 2005; 11/857,948, filed Sep. 19, 2007; 11/273,640, filed Nov. 14, 2005 and provisional patent application 60/869,295, filed Dec. 8, 2006 are incorporated by reference in their entirety.

A. Sample Preparation

While endogenous miRNA is contemplated for use with compositions and methods of the invention, recombinant miRNA—including nucleic acids that are complementary or identical to endogenous miRNA or precursor miRNA—can also be handled and analyzed as described herein. Samples may be biological samples, in which case, they can be from lavage, biopsy, fine needle aspirates, exfoliates, blood, sputum, tissue, organs, semen, saliva, tears, urine, cerebrospinal fluid, body fluids, hair follicles, skin, or any sample containing or constituting biological cells. In certain embodiments, samples may be, but are not limited to, fresh, frozen, fixed, formalin-fixed, preserved, RNAlater-preserved, paraffin-embedded, or formalin-fixed and paraffin-embedded. Alternatively, the sample may not be a biological sample, but be a chemical mixture, such as a cell-free reaction mixture (which may contain one or more biological enzymes).

B. Differential Expression Analyses

Methods of the invention can be used to detect differences in miRNA expression or levels between two samples, or a sample and a reference (e.g., a tissue reference or a digital reference representative of a non-cancerous state). Specifically contemplated applications include identifying and/or quantifying differences between miRNA from a sample that is normal and from a sample that is not normal, between a cancerous condition and a non-cancerous condition, between two differently treated samples (e.g., a pretreatment versus a post-treatment sample) or between cancerous samples with differing prognosis. Also, miRNA may be compared between a sample believed to be susceptible to a particular therapy, disease, or condition and one believed to be not susceptible or resistant to that therapy, disease, or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of a disease or condition of which a component is or may or may not be genetic or caused by a hyperproliferative or neoplastic cell or cells.

It is specifically contemplated that the invention can be used to evaluate differences between stages of disease, such as between hyperplasia, neoplasia, pre-cancer and cancer, or between a primary tumor and a metastasized tumor.

Phenotypic traits also include characteristics such as longevity, morbidity, susceptibility or receptivity to particular drugs or therapeutic treatments (drug efficacy), and risk of drug toxicity.

In certain embodiments, miRNA profiles may be generated to evaluate and correlate those profiles with pharmacokinetics. For example, miRNA profiles may be created and evaluated for patient tumor samples prior to the patient's being treated or during treatment to determine if there are miRNAs whose expression correlates with the outcome of treatment. Identification of differential miRNAs can lead to a diagnostic assay involving them that can be used to evaluate tumor samples to determine what drug regimen the patient should be provided. In addition, it can be used to identify or select patients suitable for a particular clinical trial. If a miRNA profile is determined to be correlated with drug efficacy or drug toxicity that may be relevant to whether that patient is an appropriate patient for receiving the drug or for a particular dosage of the drug.

In addition to the above assay, cancer samples from patients can be evaluated to identify a disease or a condition based on miRNA levels, such as likelihood of disease recurrence. A diagnostic assay can be created based on the profiles that doctors can use to identify individuals with a disease or who are at risk to develop a disease. Alternatively, treatments can be designed based on miRNA profiling. Examples of such methods and compositions are described in the U.S. Provisional Patent Application entitled “Methods and Compositions Involving miRNA and miRNA Inhibitor Molecules” filed on May 23, 2005, which is hereby incorporated by reference in its entirety.

C. Amplification

Many methods exist for evaluating miRNA levels by amplifying all or part of miRNA nucleic acid sequences such as mature miRNAs, precursor miRNAs, and primary miRNAs. Suitable nucleic acid polymerization and amplification techniques include reverse transcription (RT), polymerase chain reaction (PCR), real-time PCR (quantitative PCR (q-PCR)), nucleic acid sequence-base amplification (NASBA), ligase chain reaction, multiplex ligatable probe amplification, invader technology (Third Wave), rolling circle amplification, in vitro transcription (IVT), strand displacement amplification, transcription-mediated amplification (TMA), RNA (Eberwine) amplification, and other methods that are known to persons skilled in the art. In certain embodiments, more than one amplification method may be used, such as reverse transcription followed by real time PCR (Chen et al., 2005 and/or U.S. patent application Ser. No. 11/567,082, filed Dec. 5, 2006, which are incorporated herein by reference in its entirety).

A typical PCR reaction includes multiple amplification steps, or cycles that selectively amplify target nucleic acid species. A typical PCR reaction includes three steps: a denaturing step in which a target nucleic acid is denatured; an annealing step in which a set of PCR primers (forward and reverse primers) anneal to complementary DNA strands; and an elongation step in which a thermostable DNA polymerase elongates the primers. By repeating these steps multiple times, a DNA fragment is amplified to produce an amplicon, corresponding to the target DNA sequence. Typical PCR reactions include 20 or more cycles of denaturation, annealing, and elongation. In many cases, the annealing and elongation steps can be performed concurrently, in which case the cycle contains only two steps. Since mature miRNAs are single stranded, a reverse transcription reaction (which produces a complementary cDNA sequence) is performed prior to PCR reactions. Reverse transcription reactions include the use of, e.g., a RNA-based DNA polymerase (reverse transcriptase) and a primer.

In PCR and q-PCR methods, for example, a set of primers is used for each target sequence. In certain embodiments, the lengths of the primers depends on many factors, including, but not limited to, the desired hybridization temperature between the primers, the target nucleic acid sequence, and the complexity of the different target nucleic acid sequences to be amplified. In certain embodiments, a primer is about 15 to about 35 nucleotides in length. In other embodiments, a primer is equal to or fewer than 15, 20, 25, 30, or 35 nucleotides in length. In additional embodiments, a primer is at least 35 nucleotides in length.

In a further aspect, a forward primer can comprise at least one sequence that anneals to a target miRNA and alternatively can comprise an additional 5′ non-complementary region. In another aspect, a reverse primer can be designed to anneal to the complement of a reverse transcribed miRNA. The reverse primer may be independent of the miRNA sequence, and multiple miRNAs may be amplified using the same reverse primer. Alternatively, a reverse primer may be specific for a miRNA.

In some embodiments, two or more miRNAs or nucleic acids are amplified in a single reaction volume or multiple reaction volumes. In certain aspects, one or more miRNA or nucleic may be used as a normalization control or a reference nucleic acid for normalization. Normalization may be performed in separate or the same reaction volumes as other amplification reactions. One aspect includes multiplex q-PCR, such as qRT-PCR, which enables simultaneous amplification and quantification of at least one miRNA of interest and at least one reference nucleic acid in one reaction volume by using more than one pair of primers and/or more than one probe. The primer pairs comprise at least one amplification primer that uniquely binds each nucleic acid, and the probes are labeled such that they are distinguishable from one another, thus allowing simultaneous quantification of multiple miRNAs. Multiplex qRT-PCR has research and diagnostic uses, including but not limited to detection of miRNAs for diagnostic, prognostic, and therapeutic applications.

A single combined reaction for q-PCR, may be used to: (1) decrease risk of experimenter error, (2) reduce assay-to-assay variability, (3) decrease risk of target or product contamination, and (4) increase assay speed. The qRT-PCR reaction may further be combined with the reverse transcription reaction by including both a reverse transcriptase and a DNA-based thermostable DNA polymerase. When two polymerases are used, a “hot start” approach may be used to maximize assay performance (U.S. Pat. Nos. 5,411,876 and 5,985,619). For example, the components for a reverse transcriptase reaction and a PCR reaction may be sequestered using one or more thermoactivation methods or chemical alteration to improve polymerization efficiency (U.S. Pat. Nos. 5,550,044, 5,413,924, and 6,403,341).

To assess the expression of microRNAs, real-time RT-PCR detection can be used to screen nucleic acids or RNA isolated from samples of interest and a related reference such as normal adjacent tissue (NAT) samples.

A panel of amplification targets is chosen for real-time RT-PCR quantification. The selection of the panel or targets can be based on the results of microarray expression analyses, such as mirVana™ miRNA Bioarray V1, Ambion. In one aspect, the panel of targets includes one or more miRNA described herein. One example of a normalization target is 5S rRNA and others can be included. Reverse transcription (RT) reaction components are typically assembled on ice prior to the addition of RNA template. Total RNA template is added and mixed. RT reactions are incubated in an appropriate PCR System at an appropriate temperature (15-70° C., including all values and ranges there between) for an appropriate time, 15 to 30 minutes or longer, then at a temperature of 35 to 42 to 50° C. for 10 to 30 to 60 minutes, and then at 80 to 85 to 95° C. for 5 minutes, then placed on wet ice. Reverse Transcription reaction components typically include nuclease-free water, reverse transcription buffer, dNTP mix, RT Primer, RNase Inhibitor, Reverse Transcriptase, and RNA.

PCR reaction components are typically assembled on ice prior to the addition of the cDNA from the RT reactions. Following assembly of the PCR reaction components a portion of the RT reaction is transferred to the PCR mix. PCR reaction are then typically incubated in an PCR system at an elevated temperature (e.g., 95° C.) for 1 minute or so, then for a number of cycles of denaturing, annealing, and extension (e.g., 40 cycles of 95° C. for 5 seconds and 60° C. for 30 seconds). Results can be analyzed, for example, with SDS V2.3 (Applied Biosystems). Real-time PCR components typically include Nuclease-free water, MgCl₂, PCR Buffer, dNTP mix, one or more primers, DNA Polymerase, cDNA from RT reaction and one or more detectable label.

Software tools such as NormFinder (Andersen et al., 2004) are used to determine targets for normalization with the targets of interest and tissue sample set. For normalization of the real-time RT-PCR results, the cycle threshold (CO value (a log value) for the microRNA of interest is subtracted from the geometric mean Cₜ value of normalization targets. Fold change can be determined by subtracting the dCₜ normal reference (N) from the corresponding dCₜ sample being evaluated (T), producing a ddCₜ(T-N) value for each sample. The average ddCₜ(T-N) value across all samples is converted to fold change by 2^{ddct}. The representative p-values are determined by a two-tailed paired Student's t-test from the dCₜ values of sample and normal reference.

D. Nucleic Acid Arrays

Certain aspects of the present invention concern the preparation and use of miRNA arrays or miRNA probe arrays, which are ordered macroarrays or microarrays of nucleic acid molecules (probes) that are fully or nearly complementary or identical to a plurality of miRNA molecules or precursor miRNA molecules and are positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference. Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

E. Hybridization

After an array or a set of miRNA probes is prepared and the miRNA in the sample is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in many embodiments is the use of stringent conditions during hybridization. Stringent conditions are known to those of skill in the art.

### III. RNA MOLECULES

MicroRNA (“miRNA” or “miR”) molecules are generally 21 to 22 nucleotides in length, though lengths of 16 and up to 35 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) becomes part of a large complex to down-regulate a particular target gene. Examples of animal miRNAs include those that imperfectly base-pair with the target, which halts translation (Olsen et al., 1999; Seggerson et al., 2002). siRNA molecules also are processed by Dicer, but from a long, double-stranded RNA molecule. siRNAs are not naturally found in animal cells, but they can direct the sequence-specific cleavage of an mRNA target through a RNA-induced silencing complex (RISC) (Denli et al., 2003).

It is understood that a “synthetic nucleic acid” of the invention means that the nucleic acid does not have a chemical structure or sequence of a naturally occurring nucleic acid. Consequently, it will be understood that the term “synthetic miRNA” refers to a “synthetic nucleic acid” that functions in a cell or under physiological conditions as a naturally occurring miRNA.

It will be understood that the term “naturally occurring” refers to something found in an organism without any intervention by a person; it could refer to a naturally-occurring wildtype or mutant molecule.

The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the invention, a nucleic acid is isolated by virtue of it having been synthesized in vitro separate from endogenous nucleic acids in a cell. It will be understood, however, that isolated nucleic acids may be subsequently mixed or pooled together.

In certain aspects, synthetic miRNA of the invention are RNA or RNA analogs. miRNA inhibitors may be DNA or RNA, or analogs thereof. Nucleic acid based miRNA and miRNA inhibitors of the invention are collectively referred to as “synthetic nucleic acids.” In other aspects, an miRNA inhibitor can be a protein or a polypeptide that interacts with an endogenous miRNA or processing.

In some embodiments, there is a synthetic or isolated miRNA having a length of between 17 and 130 residues. The present invention concerns synthetic miRNA molecules that are, are at least, or are at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 145, 150, 160, 170, 180, 190, 200 or more residues in length, including any integer or any range derivable therein.

In certain embodiments, synthetic miRNA have (a) a “miRNA region” whose sequence from 5′ to 3′ is identical to all or a segment of a mature miRNA sequence, and (b) a “complementary region” whose sequence from 5′ to 3′ is between 60% and 100% complementary to the miRNA sequence. In certain embodiments, these synthetic miRNA are also isolated, as defined above. The term “miRNA region” refers to a region on the synthetic miRNA that is at least 75, 80, 85, 90, 95, or 100% identical, including all integers there between, to the entire sequence of a mature, naturally occurring miRNA sequence. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA. Alternatively, the miRNA region can comprise 18, 19, 20, 21, 22, 23, 24 or more nucleotide positions in common with a naturally-occurring miRNA as compared by sequence alignment algorithms and methods well known in the art.

The term “complementary region” refers to a region of a synthetic miRNA that is or is at least 60% complementary to the mature, naturally occurring miRNA sequence that the miRNA region is identical to. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there may be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments of the invention, there are synthetic nucleic acids that are miRNA inhibitors. A miRNA inhibitor is between about 17 to 25 nucleotides in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, or 25 nucleotides in length, or any range derivable therein. Moreover, a miRNA inhibitor has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. One of skill in the art could use a portion of the probe sequence that is complementary to the sequence of a mature miRNA as the sequence for a miRNA inhibitor. Moreover, that portion of the probe sequence can be altered so that it is still 90% complementary to the sequence of a mature miRNA.

In some embodiments, of the invention, a synthetic miRNA contains one or more design elements. These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or, (iii) noncomplementarity between one or more nucleotides in the last 1 to 5 residues at the 3′ end of the complementary region and the corresponding nucleotides of the miRNA region.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2′O-Me (2′ oxygen-methyl), DMTO (4,4′-dimethoxytrityl with oxygen), fluoroscein, a thiol, or acridine, though other replacement groups are well known to those of skill in the art and can be used as well. This design element can also be used with a miRNA inhibitor.

Additional embodiments concern a synthetic miRNA having one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there is one or more sugar modifications in the last 1, 2, 3, 4, 5, 6 or more residues of the complementary region, or any range derivable therein, have a sugar modification. It will be understood that the terms “first” and “last” are with respect to the order of residues from the 5′ end to the 3′ end of the region. In particular embodiments, the sugar modification is a 2′O-Me modification. In further embodiments, there is one or more sugar modifications in the first or last 2 to 4 residues of the complementary region or the first or last 4 to 6 residues of the complementary region. This design element can also be used with a miRNA inhibitor. Thus, a miRNA inhibitor can have this design element and/or a replacement group on the nucleotide at the 5′ terminus, as discussed above.

In other embodiments of the invention, there is a synthetic miRNA in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention may have one or more of the replacement, sugar modification, or noncomplementarity designs. In certain cases, synthetic RNA molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there is a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

In some embodiments of the invention, methods and compositions involving miRNA may concern miRNA and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA containing vectors, control nucleic acids, and other probes and primers. In many embodiments, miRNA are 19-24 nucleotides in length, while miRNA probes are 5, 10, 15, 19, 20, 25, 30, to 35 nucleotides in length, including all values and ranges there between, depending on the length of the processed miRNA and any flanking regions added. miRNA precursors are generally between 62 and 110 nucleotides in humans.

Nucleic acids of the invention may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or 110 contiguous nucleotides. It is further understood that the length of complementarity within a precursor miRNA or between a miRNA probe and a miRNA or a miRNA gene are such lengths. Moreover, the complementarity may be expressed as a percentage, meaning that the complementarity between a probe and its target is 90% identical or greater over the length of the probe. In some embodiments, complementarity is or is at least 90%, 95% or 100% identical. In particular, such lengths may be applied to any nucleic acid comprising a nucleic acid sequence identified in any of SEQ ID NOs disclosed herein.

The term “recombinant” may be used and this generally refers to a molecule that has been manipulated in vitro or that is a replicated or expressed product of such a molecule.

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary. miRNA probes or nucleic acids of the invention can include, can be or can be at least 60, 65, 70, 75, 80, 85, 90, 95, 96, 97, 98, 99 or 100% complementary to their target.

Nucleic acids of the invention may be made by any technique known to one of ordinary skill in the art, such as for example, chemical synthesis, enzymatic production or biological production. It is specifically contemplated that miRNA probes of the invention are chemically synthesized.

In some embodiments of the invention, miRNAs are recovered or isolated from a biological sample. The miRNA may be recombinant or it may be natural or endogenous to the cell (produced from the cell's genome). It is contemplated that a biological sample may be treated in a way so as to enhance the recovery of small RNA molecules such as miRNA. U.S. patent application Ser. No. 10/667,126 describes such methods and it is specifically incorporated by reference herein. Generally, methods involve lysing cells with a solution having guanidinium and a detergent.

A. Isolation of Nucleic Acids

Nucleic acids may be isolated using techniques well known to those of skill in the art, though in particular embodiments, methods for isolating small nucleic acid molecules, and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography. If miRNA from cells is to be used or evaluated, methods generally involve lysing the cells with a chaotropic salt (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber; typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased, such as from C.B.S. Scientific Co., Inc. or Scie-Plas.

Methods may involve the use of organic solvents and/or alcohol to isolate nucleic acids, particularly miRNA used in methods and compositions of the invention. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column has worked particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for form a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, f) capturing the miRNA molecules. Typically the sample is dried down and resuspended in a liquid and volume appropriate for subsequent manipulation.

B. Preparation of Nucleic Acids

Alternatively, nucleic acid synthesis is performed according to standard methods. See, for example, Itakura and Riggs (1980). Additionally, U.S. Pat. Nos. 4,704,362, 5,221,619, and 5,583,013 each describe various methods of preparing synthetic nucleic acids. Non-limiting examples of a synthetic nucleic acid (e.g., a synthetic oligonucleotide), include a nucleic acid made by in vitro chemically synthesis using phosphotriester, phosphite, or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al., 2001, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Recombinant methods for producing nucleic acids in a cell are well known to those of skill in the art. These include the use of vectors (viral and non-viral), plasmids, cosmids, and other vehicles for delivering a nucleic acid to a cell, which may be the target cell (e.g., a cancer cell) or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

In certain embodiments, the present invention concerns nucleic acid molecules that are not synthetic. In some embodiments, the nucleic acid molecule has a chemical structure of a naturally occurring nucleic acid and a sequence of a naturally occurring nucleic acid, such as the exact and entire sequence of a single stranded primary miRNA (see Lee, 2002), a single-stranded precursor miRNA, or a single-stranded mature miRNA. In addition to the use of recombinant technology, such non-synthetic nucleic acids may be generated chemically, such as by employing technology used for creating oligonucleotides.

C. Labels and Labeling Techniques

In some embodiments, the present invention concerns miRNA that are directly or indirectly labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In many embodiments of the invention, the label is non-radioactive. Generally, nucleic acids may be labeled by adding labeled nucleotides (one-step process) or adding nucleotides and labeling the added nucleotides (two-step process).

In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference.

In other embodiments, an unlabeled nucleotide or nucleotides is catalytically added to a miRNA, and the unlabeled nucleotide is modified with a chemical moiety that enables it to be subsequently labeled. In embodiments of the invention, the chemical moiety is a reactive amine such that the nucleotide is an amine-modified nucleotide. Examples of amine-modified nucleotides are well known to those of skill in the art, many being commercially available such as from Ambion, Sigma, Jena Bioscience, and TriLink.

In contrast to labeling of cDNA during its synthesis, the issue for labeling miRNA is how to label the already existing molecule. The present invention concerns the use of an enzyme capable of using a di- or tri-phosphate ribonucleotide or deoxyribonucleotide as a substrate for its addition to a miRNA. Moreover, in specific embodiments, it involves using a modified di- or tri-phosphate ribonucleotide, which is added to the 3′ end of a miRNA. The source of the enzyme is not limiting. Examples of sources for the enzymes include yeast, gram-negative bacteria such as E. coli, lactococcus lactis, and sheep pox virus.

Enzymes capable of adding such nucleotides include, but are not limited to, poly(A) polymerase, terminal transferase, and polynucleotide phosphorylase. In specific embodiments of the invention, a ligase is contemplated as not being the enzyme used to add the label, and instead, a non-ligase enzyme is employed.

Terminal transferase catalyzes the addition of nucleotides to the 3′ terminus of a nucleic acid.

Polynucleotide phosphorylase can polymerize nucleotide diphosphates without the need for a primer.

Labels on miRNA or miRNA probes may be colorimetric (includes visible and UV spectrum, including fluorescent), luminescent, enzymatic, or positron emitting (including radioactive). The label may be detected directly or indirectly. Radioactive labels include ¹²⁵I, ³²P, ³³P, and ³⁵S. Examples of enzymatic labels include alkaline phosphatase, luciferase, horseradish peroxidase, and β-galactosidase. Labels can also be proteins with luminescent properties, e.g., green fluorescent protein and phycoerythrin.

The colorimetric and fluorescent labels contemplated for use as conjugates include, but are not limited to, Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides are available from Molecular Probes, and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences, such as Cy3-UTP and Cy5-UTP.

Examples of fluorescently labeled deoxyribonucleotides include Dinitrophenyl (DNP)-11-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP.

It is contemplated that nucleic acids may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference).

Alternatively, the label may not be detectable per se, but indirectly detectable or allowing for the isolation or separation of the targeted nucleic acid. For example, the label could be biotin, digoxigenin, polyvalent cations, chelator groups and the other ligands, include ligands for an antibody.

A number of techniques for visualizing or detecting labeled nucleic acids are readily available. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC (Griffey et al., 1997), spectroscopy, capillary gel electrophoresis (Cummins et al., 1996), spectroscopy; mass spectroscopy; radiological techniques; and mass balance techniques.

When two or more differentially colored labels are employed, fluorescent resonance energy transfer (FRET) techniques may be employed to characterize association of one or more nucleic acid. Furthermore, a person of ordinary skill in the art is well aware of ways of visualizing, identifying, and characterizing labeled nucleic acids, and accordingly, such protocols may be used as part of the invention. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

### IV. KITS

Any of the compositions or components described herein may be comprised in a kit. In a non-limiting example, reagents for isolating miRNA, labeling miRNA, and/or evaluating a miRNA population using an array, nucleic acid amplification, and/or hybridization can be included in a kit, as well reagents for preparation of samples from colorectal samples. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. In certain aspects, the kit can include amplification reagents. In other aspects, the kit may include various supports, such as glass, nylon, polymeric beads, magnetic beads, and the like, and/or reagents for coupling any probes and/or target nucleic acids. It may also include one or more buffers, such as reaction buffer, labeling buffer, washing buffer, or a hybridization buffer, compounds for preparing the miRNA probes, and components for isolating miRNA. Other kits of the invention may include components for making a nucleic acid array comprising miRNA, and thus, may include, for example, a solid support.

Kits for implementing methods of the invention described herein are specifically contemplated. In some embodiments, there are kits for preparing miRNA for multi-labeling and kits for preparing miRNA probes and/or miRNA arrays. In these embodiments, kit comprise, in suitable container means, 1, 2, 3, 4, 5, 6, 7, 8, 9, 10, 11, 12 or more of the following: (1) poly(A) polymerase; (2) unmodified nucleotides (G, A, T, C, and/or U); (3) a modified nucleotide (labeled or unlabeled); (4) poly(A) polymerase buffer; and, (5) at least one microfilter; (6) label that can be attached to a nucleotide; (7) at least one miRNA probe; (8) reaction buffer; (9) a miRNA array or components for making such an array; (10) acetic acid; (11) alcohol; (12) solutions for preparing, isolating, enriching, and purifying miRNAs or miRNA probes or arrays. Other reagents include those generally used for manipulating RNA, such as formamide, loading dye, ribonuclease inhibitors, and DNase.

In specific embodiments, kits of the invention include an array containing miRNA probes, as described in the application. An array may have probes corresponding to all known miRNAs of an organism or a particular tissue or organ in particular conditions, or to a subset of such probes. The subset of probes on arrays of the invention may be or include those identified as relevant to a particular diagnostic, therapeutic, or prognostic application. For example, the array may contain one or more probes that is indicative or suggestive of (1) a disease or condition (colorectal cancer), (2) susceptibility or resistance to a particular drug or treatment; (3) susceptibility to toxicity from a drug or substance; (4) the stage of development or severity of a disease or condition (one aspect of prognosis); (5) the likelihood of cancer recurrence (one aspect of prognosis) and (6) genetic predisposition to a disease or condition.

For any kit embodiment, including an array, there can be nucleic acid molecules that contain or can be used to amplify a sequence that is a variant of, identical to or complementary to all or part of any of SEQ ID NOs described herein. Any nucleic acid discussed above may be implemented as part of a kit.

The components of the kits may be packaged either in aqueous media or in lyophilized form. The container means of the kits will generally include at least one vial, test tube, flask, bottle, syringe or other container means, into which a component may be placed, and preferably, suitably aliquotted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be resuspended in any suitable solvent, such as DMSO.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Such kits may also include components that facilitate isolation of the labeled miRNA. It may also include components that preserve or maintain the miRNA or that protect against its degradation. Such components may be RNase-free or protect against RNases. Such kits generally will comprise, in suitable means, distinct containers for each individual reagent or solution.

A kit will also include instructions for employing the kit components as well the use of any other reagent not included in the kit. Instructions may include variations that can be implemented.

Kits of the invention may also include one or more of the following: Control RNA; nuclease-free water; RNase-free containers, such as 1.5 ml tubes; RNase-free elution tubes; PEG or dextran; ethanol; acetic acid; sodium acetate; ammonium acetate; guanidinium; detergent; nucleic acid size marker; RNase-free tube tips; and RNase or DNase inhibitors.

It is contemplated that such reagents are embodiments of kits of the invention. Such kits, however, are not limited to the particular items identified above and may include any reagent used for the manipulation or characterization of miRNA.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Microarray Identification of MicroRNA Biomarkers for Colorectal Cancer

miRNAs potentially relevant to carcinogenesis frequently exhibit differential expression in cancer versus normal samples collected from the same tissue type. In addition, miRNAs with differential expression in normal and cancerous samples may be used in the diagnosis of pre-cancerous and cancerous lesions and in patient prognosis. To identify miRNAs that may be useful markers for diagnosis of colorectal cancer and for establishing patient prognosis, the inventors evaluated miRNA expression in twenty cancerous and twenty paired normal adjacent tissue (NAT) samples from the same patients (Table 2).

TABLE 2
Histopathological data and patient information for colorectal cancer tissue (D) and
normal adjacent tissue (NAT) samples. R, recurrent; NR, non-recurrent; NA, not available

| | Patient | | | | | | | | |
|---|---|---|---|---|---|---|---|---|---|
| | Information | | Clinical Stage | Node Status | Tumor Status | | Cancer | Time to | Distant |
| | Age In | | (Greene | (Greene | (Greene | Tissue | Recurrence | Recurrence | Metastasis |
| Sample | Years | Gender | et al., 2002) | et al., 2002) | et al., 2002) | type | Status | (months) | Site |
| D1415 | 58 | M | I | 0 | 2 | Recto- | R | 20 | NA |
| NAT1415 | | | | | | sigmoid | | | |
| D1416 | 70 | F | I | 0 | 2 | Colon | NR | | |
| NAT1416 | | | | | | | | | |
| D1417 | 54 | NA | IIA | 0 | 3 | Colon | R | 5 | Liver |
| NAT1417 | | | | | | | | | |
| D1418 | 86 | F | IIA | 0 | 3 | Colon | NR | | |
| NAT1418 | | | | | | | | | |
| D1419 | 70 | F | IIA | 0 | 3 | Colon | R | 24 | Lymph |
| NAT1419 | | | | | | | | | Nodes |
| D1420 | 50 | F | IIA | 0 | 3 | Cecum | NR | | |
| NAT1420 | | | | | | | | | |
| D1421 | 77 | F | IIA | 0 | 3 | Colon | R | 26 | Lung |
| NAT1421 | | | | | | | | | |
| D1422 | 80 | M | IIA | 0 | 3 | Colon | NR | | |
| NAT1422 | | | | | | | | | |
| D1423 | 73 | M | IIIB | 1 | 3 | Colon | R | 12 | Colon |
| NAT1423 | | | | | | | | | |
| D1424 | 32 | F | IIIB | 1 | 3 | Colon | NR | | |
| NAT1424 | | | | | | | | | |
| D1425 | 82 | M | IIIB | 1 | 3 | Colon | R | 17 | Lung |
| NAT1425 | | | | | | | | | |
| D1426 | 78 | F | IIIB | 1 | 3 | Colon | NR | | |
| NAT1426 | | | | | | | | | |
| D1427 | 71 | F | IIIB | 1 | 3 | Recto- | R | 24 | Liver |
| NAT1427 | | | | | | sigmoid | | | |
| D1428 | 48 | M | IIIB | 1 | 3 | Colon | NR | | |
| NAT1428 | | | | | | | | | |
| D1429 | 60 | F | IIA | 0 | 3 | Colon | R | 24 | Ovary |
| NAT1429 | | | | | | | | | |
| D1430 | 47 | M | IIA | 0 | 3 | Colon | NR | | |
| NAT1430 | | | | | | | | | |
| D1431 | 76 | M | IIIA | 1 | 2 | Colon | R | 24 | Lung |
| NAT1431 | | | | | | | | | |
| D1432 | 88 | F | IIIA | 1 | 2 | Colon | NR | | |
| NAT1432 | | | | | | | | | |
| D1433 | 46 | M | IIA | 0 | 3 | Recto- | R | 60 | Rectum |
| NAT1433 | | | | | | sigmoid | | | |
| D1434 | 75 | F | IIA | 0 | 3 | Cecum | NR | | |
| NAT1434 | | | | | | | | | |

Formalin-fixed, paraffin-embedded (FFPE) tissue specimens from mixed stage colorectal tumors and normal adjacent tissues from the same patient (Table 2) were acquired using appropriate human subjects regulations. Colorectal cancer and normal adjacent tissue specimens were from two groups of patients; those with documented recurrence of colorectal cancer within five years of initial surgery to remove a primary tumor and those without documented cancer recurrence within five years from initial surgery, but having been monitored for 5 years. To assure sample type and integrity, all samples were evaluated by a board-certified anatomic pathologist. Prior to RNA isolation, FFPE cancerous tissue samples were macro-dissected to maximize the percentage of cancer cells present for RNA isolation. All macro-dissected samples contained a minimum of 90% cancer cells prior to RNA isolation.

Total RNA isolation from tissue samples was performed using a RecoverAll™ kit for use with FFPE tissues (Ambion, Inc.; Austin, Tex., USA). MicroRNA-containing fractions were purified from 10 μg of total RNA using a flashPAGE™ Fractionation Apparatus (Ambion). Purified small RNAs were enzymatically biotinylated at their 3′-termini using the mirVana™ miRNA Labeling Kit (Ambion). The dNTP mixture provided in the mirVana™ kit for the tailing reaction was replaced with a proprietary nucleotide mixture containing biotin-modified nucleotides (PerkinElmer, Waltham, Mass., USA). Labeled miRNAs were hybridized to a custom-made microRNA array by Asuragen Services (Asuragen, Inc., Austin, Tex., USA). The microarray contained probes for 1208 miRNAs including those from the Sanger miRBase v9.2 database (Griffiths-Jones et al., 2006), and published reports (Berezikov et al., 2005), (Xie et al., 2005). Hybridization, washing, staining, imaging, and signal extraction were performed as recommended by the microarray manufacturer, except that the 20× GeneChip@ Eukaryotic Hybridization Control cocktail was omitted.

Signal processing with the custom microarray is a multi-step process involving probe-specific signal detection calls, background estimate and correction, constant variance stabilization and either array scaling or global normalization. For an overview of miRNA processing and analysis see Davison et al. (2006).

Probe Specific Signal Detection Calls.

Each probe on the array was assayed for detection based on a Wilcoxon rank-sum test of the miRNA probe signal compared to the distribution of signals from GC-content-matched, anti-genomic probes. If the resulting p-value for the probes was ≦0.06 then it was considered “detected above background.” Probes with p-values>0.06 had insufficient signal to discriminate from the background and were thus considered not detected.

Background Estimate and Correction.

The same set of anti-genomic probes used to determine detection calls were used to estimate GC-content-matched background signals. Each miRNA probe signal had a GC-content-matched background estimate subtracted from its value. This GC-specific background contribution was estimated by the median signal from the distribution of GC-matched, anti-genomic probes.

Constant Variance Stabilization.

Probes with low signal often exhibited a high degree of variability once transformed into logarithmic scales. To stabilize this low signal variability, the inventors added a constant value of 16 to the background corrected signal, a technique commonly performed on microarray data and the recommended data preprocessing method by Affymetrix and Illumina in the Microarray Quality Control (MAQC) project (MAQC Consortium, 2006).

Normalization.

The data were normalized with the VSN method (Huber et al., 2002). VSN is a global normalization process that stabilizes the variance evenly across the entire range of expression. Differences in VSN-transformed expression between samples are denoted by “log 2Diff” and can be transformed to a generalized fold change via exponentiation base 2. These values will exhibit a compression for small differences in expression.

Statistical Hypothesis Testing.

For statistical hypothesis testing, a two-sample t-test, with assumption of equal variance, was applied. One-way ANOVA was used for experimental designs with more than two experimental groupings or levels of the same factor. These tests define which miRNAs were considered to be significantly differentially expressed based on a default p-value of 0.05 and log₂ difference>1.

Results of the miRNA expression analysis in paired colorectal cancer tissue and normal adjacent tissue samples are shown below in Table 3.

Differentially-expressed miRNAs in Table 3 that have not been identified in previously published reports (Akao et al., 2006; Akao et al., 2007; Bandres et al., 2006, Cummins et al., 2006; Xi et al., 2006a, 2006b) are shown below in Table 4.

TABLE 3
MicroRNAs Differentially Expressed Between Colorectal Cancer
Tissue Samples (Tumor) and Normal Adjacent Tissue Samples
(NAT) from Patients by Microarray Quantification. Mean, mean
of normalized array data for 20 tissue samples; Log2Diff,
difference in VSN-transformed expression between NAT and
Tumor samples.

| | Mean | Mean | Log2 diff | Fold | |
|---|---|---|---|---|---|
| miRNA | NAT | Tumor | NAT-Tumor | Change | t-test |
| hsa-miR-215 | 10.07 | 7.53 | 2.54 | 5.82 | 8.19E−05 |
| hsa-miR-451 | 8.81 | 6.79 | 2.02 | 4.06 | 7.70E−05 |
| hsa-miR-422a | 9.05 | 7.04 | 2.01 | 4.04 | 3.43E−06 |
| hsa-miR-422b | 10.84 | 8.85 | 1.99 | 3.98 | 6.41E−06 |
| hsa-miR-133b | 6.89 | 5.07 | 1.82 | 3.52 | 4.86E−03 |
| hsa-miR-133a | 7.68 | 5.89 | 1.79 | 3.47 | 1.72E−03 |
| hsa-miR-195 | 10.50 | 8.74 | 1.76 | 3.39 | 3.20E−03 |
| hsa-miR-194 | 11.88 | 10.21 | 1.67 | 3.19 | 8.00E−03 |
| hsa-miR43 | 6.27 | 4.69 | 1.58 | 2.98 | 4.10E−03 |
| hsa-miR-30c | 9.45 | 7.95 | 1.50 | 2.82 | 8.32E−03 |
| hsa-miR-192 | 12.44 | 11.07 | 1.36 | 2.57 | 7.41E−03 |
| hsa-miR-497 | 6.20 | 4.86 | 1.35 | 2.54 | 4.51E−03 |
| hsa-miR-1 | 8.63 | 7.30 | 1.32 | 2.50 | 1.97E−02 |
| hsa-miR-375 | 9.47 | 8.15 | 1.32 | 2.49 | 1.35E−02 |
| hsa-miR-145 | 12.06 | 10.79 | 1.27 | 2.41 | 3.16E−02 |
| hsa-miR-150 | 8.76 | 7.49 | 1.27 | 2.41 | 3.59E−02 |
| hsa-miR-30b | 8.85 | 7.69 | 1.16 | 2.23 | 2.32E−02 |
| hsa-cand342 | 8.16 | 9.19 | −1.03 | 2.04 | 9.46E−03 |
| hsa-miR-183 | 3.44 | 4.50 | −1.06 | 2.08 | 1.54E−02 |
| hsa-miR-182 | 6.14 | 7.22 | −1.07 | 2.10 | 2.89E−02 |
| hsa-miR30 | 4.00 | 5.09 | −1.10 | 2.14 | 1.45E−02 |
| hsa-miR-224 | 4.52 | 5.63 | −1.11 | 2.16 | 3.64E−02 |
| hsa-miR-31 | 4.60 | 7.59 | −2.99 | 7.94 | 2.29E−03 |
| hsa-miR-143 | 11.63 | 10.79 | 0.84 | 1.79 | 4.41E−02 |
| hsa-miR-30a-5p | 8.73 | 7.73 | 1.00 | 2.00 | 5.49E−03 |
| hsa-cand26 | 4.14 | 3.14 | 1.00 | 2.00 | 2.11E−03 |
| hsa-miR-10b | 9.77 | 8.78 | 0.99 | 1.99 | 5.72E−03 |
| hsa-miR-30e-5p | 7.36 | 6.42 | 0.94 | 1.91 | 8.20E−03 |

TABLE 4
Novel MicroRNAs Differentially Expressed Between Colorectal
Cancer Tissue Samples (Tumor) and Normal Adjacent Tissue
Samples (NAT) from Patients. Mean, mean of normalized array
data for 20 tissue samples; Log2Diff, difference in VSN-
transformed expression between NAT and Tumor samples.
Negative Log2 diff values indicate miRNAs expressed at higher
levels in tumor samples.

| | Mean | Mean | Log2Diff | Fold | |
|---|---|---|---|---|---|
| miRNA | NAT | Tumor | NAT-Tumor | Change | t-test |
| hsa-miR-451 | 8.81 | 6.79 | 2.02 | 4.06 | 7.70E−05 |
| hsa-miR-422a | 9.05 | 7.04 | 2.01 | 4.04 | 3.43E−06 |
| hsa-miR-195 | 10.50 | 8.74 | 1.76 | 3.39 | 3.20E−03 |
| hsa-miR-194 | 11.88 | 10.21 | 1.67 | 3.19 | 8.00E−03 |
| hsa-miR43 | 6.27 | 4.69 | 1.58 | 2.98 | 4.10E−03 |
| hsa-miR-192 | 12.44 | 11.07 | 1.36 | 2.57 | 7.41E−03 |
| hsa-miR-497 | 6.20 | 4.86 | 1.35 | 2.54 | 4.51E−03 |
| hsa-miR-1 | 8.63 | 7.30 | 1.32 | 2.50 | 1.97E−02 |
| hsa-miR-375 | 9.47 | 8.15 | 1.32 | 2.49 | 1.35E−02 |
| hsa-miR-150 | 8.76 | 7.49 | 1.27 | 2.41 | 3.59E−02 |
| hsa-miR-30b | 8.85 | 7.69 | 1.16 | 2.23 | 2.32E−02 |
| hsa-cand342 | 8.16 | 9.19 | −1.03 | 2.04 | 9.46E−03 |
| hsa-miR30 | 4.00 | 5.09 | −1.10 | 2.14 | 1.45E−02 |
| hsa-miR-224 | 4.52 | 5.63 | −1.11 | 2.16 | 3.64E−02 |
| hsa-miR-30a-5p | 8.73 | 7.73 | 1.00 | 2.00 | 5.49E−03 |
| hsa-cand26 | 4.14 | 3.14 | 1.00 | 2.00 | 2.11E−03 |
| hsa-miR-10b | 9.77 | 8.78 | 0.99 | 1.99 | 5.72E−03 |
| hsa-miR-30e-5p | 7.36 | 6.42 | 0.94 | 1.91 | 8.20E−03 |

The microRNAs in Table 4 represent particularly useful markers for diagnosing colorectal cancer. Comparing the expression levels of these specific miRNAs in a colorectal tissue sample that is suspected of being cancerous with their expression levels in a reference non-cancerous colorectal tissue sample indicates whether or not the suspect tissue is cancerous.

### Example 2

### Identification of MicroRNA Biomarkers for Colorectal Cancer Recurrence from Mixed-Stage Tumors

To identify miRNAs that are useful for predicting colorectal cancer recurrence in cancer patients, the inventors used microarray quantification to determine miRNAs that are differentially expressed between tumors from ten patients with recurrent colorectal cancer and tumors from ten patients that did not have recurrence within a five year period (Table 2). miRNA isolation and purification and microarray quantification were performed as described above in Example 1. Results of the miRNA expression analysis are shown below in Table 5. All miRNAs that were expressed at significantly different levels between the two tumor groups were expressed at lower average levels in the tumors from patients with recurrent colorectal cancer. The inventors found no significant differences in miRNA expression between normal adjacent tissue samples from patients with recurrent colon cancer and normal adjacent tissue samples from patients that did not have cancer recurrence.

TABLE 5
MicroRNAs Differentially Expressed Between Mixed-Stage
Tumors from Patients with and without Colorectal Cancer
Recurrence. Mean, mean of normalized array data for 10
tissue samples from patients with (recurrent) or without
(non-recurrent) cancer recurrence; Log2Diff, difference in
VSN-transformed expression between tumor samples from
patients with (recurrent) or without (non-recurrent) cancer
recurrence.

| | | | Log2Diff | | ttest |
|---|---|---|---|---|---|
| | | | non- | | non- |
| | Mean | Mean | recurrent | | recurrent |
| | non- | re- | vs | Fold | vs |
| miRNA | recurrent | current | recurrent | Change | recurrent |
| hsa-miR-1 | 8.45 | 6.26 | 2.18 | 4.54 | 0.016 |
| hsa-miR-20a | 9.90 | 7.83 | 2.07 | 4.20 | 0.020 |
| hsa-miR-194 | 11.28 | 9.29 | 1.98 | 3.95 | 0.028 |
| hsa-miR-203 | 10.10 | 8.15 | 1.95 | 3.86 | 0.028 |
| hsa-miR-26b | 10.29 | 8.48 | 1.81 | 3.50 | 0.019 |
| hsa-miR-15a | 7.89 | 6.08 | 1.81 | 3.50 | 0.006 |
| hsa-miR-133b | 6.04 | 4.26 | 1.79 | 3.45 | 0.020 |
| hsa-miR-107 | 10.18 | 8.41 | 1.77 | 3.40 | 0.024 |
| hsa-miR-141 | 8.20 | 6.47 | 1.73 | 3.32 | 0.028 |
| hsa-miR-155 | 10.12 | 8.40 | 1.73 | 3.31 | 0.009 |
| hsa-miR-20b | 8.32 | 6.60 | 1.72 | 3.30 | 0.025 |
| hsa-miR-195 | 9.89 | 8.18 | 1.71 | 3.26 | 0.049 |
| hsa-miR-106a | 9.21 | 7.52 | 1.69 | 3.22 | 0.036 |
| hsa-miR-29b | 7.27 | 5.58 | 1.68 | 3.21 | 0.005 |
| hsa-miR-223 | 8.79 | 7.12 | 1.67 | 3.18 | 0.018 |
| hsa-miR-17-5p | 9.18 | 7.53 | 1.65 | 3.13 | 0.039 |
| hsa-miR-103 | 10.36 | 8.72 | 1.63 | 3.11 | 0.022 |
| hsa-miR-660 | 6.51 | 4.89 | 1.63 | 3.09 | 0.002 |
| hsa-let-7g | 11.22 | 9.63 | 1.58 | 3.00 | 0.033 |
| hsa-miR-15b | 5.97 | 4.40 | 1.58 | 2.99 | 0.013 |
| hsa-miR-23a | 11.55 | 9.99 | 1.57 | 2.96 | 0.049 |
| hsa-miR-182 | 7.94 | 6.39 | 1.55 | 2.93 | 0.024 |
| hsa-miR-29a | 10.54 | 8.99 | 1.55 | 2.93 | 0.023 |
| hsa-miR-98 | 8.20 | 6.67 | 1.53 | 2.89 | 0.032 |
| hsa-miR-16 | 11.52 | 10.01 | 1.52 | 2.86 | 0.031 |
| hsa-miR43 | 5.65 | 4.14 | 1.51 | 2.85 | 0.041 |
| hsa-miR-106b | 7.36 | 5.88 | 1.48 | 2.79 | 0.021 |
| hsa-miR-30b | 8.68 | 7.21 | 1.47 | 2.77 | 0.048 |
| hsa-miR-27a | 9.89 | 8.43 | 1.46 | 2.75 | 0.027 |
| hsa-miR-19b | 6.13 | 4.67 | 1.46 | 2.74 | 0.012 |
| hsa-miR-27b | 10.13 | 8.70 | 1.43 | 2.70 | 0.032 |
| hsa-miR-342 | 8.92 | 7.50 | 1.43 | 2.69 | 0.027 |
| hsa-miR-146a | 10.21 | 8.80 | 1.42 | 2.67 | 0.007 |
| hsa-miR-361 | 8.71 | 7.32 | 1.38 | 2.61 | 0.047 |
| hsa-miR-93 | 8.87 | 7.49 | 1.38 | 2.60 | 0.031 |
| hsa-miR257 | 6.43 | 5.07 | 1.36 | 2.57 | 0.040 |
| hsa-miR-130a | 7.56 | 6.21 | 1.35 | 2.55 | 0.031 |
| hsa-miR-152 | 7.68 | 6.34 | 1.34 | 2.52 | 0.024 |
| hsa-miR-335 | 5.91 | 4.60 | 1.31 | 2.49 | 0.015 |
| hsa-miR-143 | 11.57 | 10.28 | 1.29 | 2.44 | 0.044 |
| hsa-miR-28 | 7.37 | 6.08 | 1.28 | 2.44 | 0.019 |
| hsa-miR-30e-5p | 7.16 | 5.91 | 1.24 | 2.37 | 0.024 |
| hsa-miR-25 | 8.78 | 7.58 | 1.21 | 2.31 | 0.041 |
| hsa-miR-146b | 10.13 | 8.94 | 1.19 | 2.28 | 0.020 |
| hsa-cand144 | 7.37 | 6.19 | 1.18 | 2.26 | 0.046 |
| hsa-miR-95 | 5.20 | 4.02 | 1.17 | 2.25 | 0.028 |
| hsa-miR-218 | 5.70 | 4.54 | 1.16 | 2.24 | 0.037 |
| hsa-miR-128a | 5.07 | 3.92 | 1.15 | 2.21 | 0.018 |
| hsa-let-7i | 11.20 | 10.06 | 1.14 | 2.20 | 0.045 |
| hsa-miR-34a | 6.96 | 5.82 | 1.14 | 2.20 | 0.029 |
| hsa-miR-130b | 6.85 | 5.73 | 1.12 | 2.18 | 0.046 |
| hsa-miR-21 | 13.13 | 12.01 | 1.11 | 2.16 | 0.044 |
| hsa-miR-30a-5p | 8.39 | 7.32 | 1.07 | 2.09 | 0.047 |
| hsa-miR-30a-3p | 5.98 | 4.92 | 1.06 | 2.09 | 0.043 |
| hsa-miR-652 | 6.44 | 5.42 | 1.03 | 2.04 | 0.015 |
| hsa-miR-625 | 6.24 | 5.21 | 1.03 | 2.04 | 0.010 |
| hsa-miR-191 | 10.92 | 9.94 | 0.98 | 1.97 | 0.039 |
| hsa-miR-17-3p | 5.47 | 4.50 | 0.97 | 1.96 | 0.040 |
| hsa-miR-222 | 7.72 | 6.75 | 0.97 | 1.96 | 0.032 |
| hsa-miR-594 | 9.21 | 8.26 | 0.95 | 1.93 | 0.043 |

The miRNAs in Table 5 represent useful prognostic markers for predicting if colorectal cancer patients are likely to have cancer recurrence within a five year period. Comparing the expression levels of these specific miRNAs in a colorectal tissue sample from a patient with colorectal cancer with their expression levels in a reference colorectal tissue sample (e.g., a sample from a patient that was known to not have cancer recurrence) or to a reference control miRNA is useful for predicting the likelihood of cancer recurrence.

### Example 3

### Analysis of Stage II and Stage III Colorectal Cancer Tumors and Identification of MicroRNA Biomarkers for Cancer Recurrence

The inventors sought to identify miRNAs that that are useful for predicting colorectal cancer recurrence in cancer patients with Stage II or Stage III cancer. Microarray quantification was used to determine miRNAs that are differentially expressed between tumors from five patients with Stage II cancer who had cancer recurrence and tumors from five patients with Stage II cancer who did not have cancer recurrence. Tumor samples have been described in Table 2 above. miRNA isolation and purification and microarray quantification were performed as described above in Example 1. Results of the miRNA expression analyses are shown below in Table 6.

TABLE 6
MicroRNAs Differentially Expressed Between Tumor Samples
from Patients with Stage II Colorectal Cancer (recurrent
vs non-recurrent). Mean, mean of normalized array data for
five tumor samples from patients with (recurrent) or with-
out (non-recurrent) cancer recurrence. Log2Diff, difference
in VSN-transformed expression between tumor samples from
patients with (recurrent) or without (non-recurrent) cancer
recurrence.

| | | | Log2Diff | | t-test |
|---|---|---|---|---|---|
| | | | Stage II | | Stage II |
| | Mean | | non- | | non- |
| | Stage II | Mean | recurrent | | recurrent |
| | non- | Stage II | vs | Fold | vs |
| miRNA | recurrent | recurrent | recurrent | Change | recurrent |
| hsa-miR-20a | 9.49 | 7.28 | 2.20 | 4.61 | 0.030 |
| hsa-miR-196b | 7.14 | 5.02 | 2.12 | 4.33 | 0.012 |
| hsa-miR-196a | 8.23 | 6.20 | 2.03 | 4.08 | 0.027 |
| hsa-miR-155 | 9.97 | 8.02 | 1.95 | 3.87 | 0.004 |
| hsa-miR-194 | 10.91 | 8.96 | 1.95 | 3.87 | 0.043 |
| hsa-miR-7 | 8.48 | 6.55 | 1.93 | 3.82 | 0.050 |
| hsa-miR-98 | 8.01 | 6.11 | 1.90 | 3.74 | 0.015 |
| hsa-miR-106a | 8.86 | 6.98 | 1.88 | 3.68 | 0.027 |
| hsa-miR-182 | 7.92 | 6.04 | 1.88 | 3.67 | 0.008 |
| hsa-miR-26b | 9.95 | 8.08 | 1.87 | 3.66 | 0.029 |
| hsa-miR-17-5p | 8.82 | 6.95 | 1.87 | 3.65 | 0.038 |
| hsa-miR-15a | 7.56 | 5.75 | 1.81 | 3.50 | 0.023 |
| hsa-miR-146a | 10.24 | 8.45 | 1.79 | 3.46 | 0.003 |
| hsa-miR-20b | 7.82 | 6.04 | 1.78 | 3.43 | 0.053 |
| hsa-miR-148a | 7.09 | 5.41 | 1.68 | 3.20 | 0.032 |
| hsa-miR-106b | 7.14 | 5.51 | 1.63 | 3.11 | 0.025 |
| hsa-miR-15b | 5.71 | 4.10 | 1.62 | 3.07 | 0.039 |
| hsa-miR-660 | 6.14 | 4.53 | 1.61 | 3.06 | 0.012 |
| hsa-miR-29b | 6.94 | 5.35 | 1.59 | 3.01 | 0.031 |
| hsa-miR-335 | 5.69 | 4.12 | 1.57 | 2.97 | 0.026 |
| hsa-miR-93 | 8.73 | 7.17 | 1.56 | 2.94 | 0.019 |
| hsa-miR-107 | 9.82 | 8.27 | 1.56 | 2.94 | 0.032 |
| hsa-let-7g | 10.95 | 9.40 | 1.54 | 2.91 | 0.032 |
| hsa-miR-19b | 5.77 | 4.23 | 1.54 | 2.90 | 0.029 |
| hsa-miR-25 | 8.62 | 7.10 | 1.52 | 2.86 | 0.018 |
| hsa-miR-29a | 10.37 | 8.88 | 1.49 | 2.81 | 0.016 |
| hsa-miR-152 | 7.36 | 5.88 | 1.48 | 2.79 | 0.032 |
| hsa-miR-103 | 10.03 | 8.58 | 1.45 | 2.74 | 0.039 |
| hsa-miR-146b | 10.13 | 8.70 | 1.43 | 2.69 | 0.010 |
| hsa-miR-128a | 4.83 | 3.44 | 1.38 | 2.61 | 0.010 |
| hsa-let-7f | 11.77 | 10.41 | 1.36 | 2.57 | 0.041 |
| hsa-miR-16 | 11.34 | 9.99 | 1.35 | 2.55 | 0.030 |
| hsa-miR-34a | 6.79 | 5.46 | 1.33 | 2.52 | 0.039 |
| hsa-miR-218 | 5.57 | 4.24 | 1.33 | 2.52 | 0.043 |
| hsa-miR-222 | 7.67 | 6.38 | 1.29 | 2.44 | 0.016 |
| hsa-miR-28 | 7.01 | 5.76 | 1.25 | 2.37 | 0.041 |
| hsa-miR-221 | 10.09 | 8.95 | 1.15 | 2.22 | 0.029 |
| hsa-miR-652 | 6.20 | 5.09 | 1.11 | 2.16 | 0.007 |
| hsa-miR-181d | 4.87 | 3.77 | 1.10 | 2.14 | 0.005 |
| hsa-let-7i | 10.98 | 9.91 | 1.07 | 2.10 | 0.029 |
| hsa-miR-191 | 10.66 | 9.68 | 0.98 | 1.97 | 0.038 |
| hsa-miR-185 | 7.68 | 6.73 | 0.94 | 1.92 | 0.046 |

miRNAs with significantly different average expression values between patients with recurrent and non-recurrent Stage II colorectal cancer were all expressed at lower average levels in patients who had cancer recurrence. The miRNAs identified in Table 6 could be used as prognostic markers to predict if patients with Stage II colorectal cancer are likely to have cancer recurrence within a five year period. Comparing the expression levels of these specific miRNAs in a colorectal tissue sample from a patient with Stage II colorectal cancer with their expression levels in a reference colorectal tissue sample (e.g., a sample from a patient with Stage II cancer that was known to not have cancer recurrence) or to a reference control miRNA is useful for predicting the likelihood of cancer recurrence.

Similarly, the inventors determined miRNAs that are differentially expressed between tumors from four patients with Stage III cancer who had cancer recurrence and tumors from four patients with Stage III cancer who did not have cancer recurrence. Tumor samples have been described in Table 2 above. miRNA isolation and purification and microarray quantification were performed as described above in Example 1. Results of this miRNA expression analyses are shown below in Table 7.

TABLE 7
MicroRNAs Differentially Expressed Between Tumor Samples
from Patients with Stage III Colorectal Cancer (recurrent vs non-
recurrent). Mean, mean of normalized array data for four tumor
samples from patients with (recurrent) or without (non-
recurrent) cancer recurrence. Log2Diff, difference in VSN-
transformed expression between tumor samples from patients
with (recurrent) or without (non-recurrent) cancer recurrence.

| | | | Log2Diff | | ttest- |
|---|---|---|---|---|---|
| | | | Stage III | | Stage III |
| | Mean | | non- | | non- |
| | Stage III | Mean | recurrent | | recurrent |
| | non- | Stage III | vs | Fold | vs |
| miRNA | recurrent | recurrent | recurrent | Change | recurrent |
| hsa-miR-133a | 7.84 | 5.44 | 2.40 | 5.28 | 0.0435 |
| hsa-miR-133b | 6.91 | 4.34 | 2.57 | 5.94 | 0.0278 |
| hsa-miR-205 | 3.29 | 2.09 | 1.20 | 2.30 | 0.0218 |
| hsa-cand173 | 4.62 | 5.65 | −1.03 | 2.04 | 0.0043 |

Among miRNAs having significantly different average expression values between patients with recurrent and non-recurrent Stage III colorectal cancer, three were expressed at lower average levels in patients who had cancer recurrence, and one was expressed at a higher average level in patients who had cancer recurrence. The miRNAs identified in Table 7 are useful as prognostic markers to predict if patients with Stage III colorectal cancer are likely to have cancer recurrence within a five year period. Comparing the expression levels of these specific miRNAs in a colorectal tissue sample from a patient with Stage III colorectal cancer with their expression levels in a reference colorectal tissue sample (e.g., a sample from a patient with Stage III colorectal cancer that was known to not have cancer recurrence) or with a reference control miRNA are also useful for predicting the likelihood of cancer recurrence. Because all Stage III colorectal cancer patients typically receive adjuvant therapy, the miRs identified in Table 7 could also be used as markers to identify patients that are responding or are not responding to adjuvant therapy. Patients with Stage III colorectal cancer who experience recurrence within five years of initial surgery represent poor responders to adjuvant therapy while Stage III patients without cancer recurrence within five years represent good responders to adjuvant therapy. Because hsa-cand173 is expressed at higher levels in patients with cancer recurrence, inhibitors of that miRNA represent useful therapeutic targets for the prevention of colorectal cancer recurrence.

The inventors also compared miRNA expression in tumors from patients with Stage II and Stage III colorectal cancer who did not have cancer recurrence within a five year period. No miRNAs were expressed at significantly different levels in these two groups. These data indicate that the miRNA profiles of colorectal cancer tumors is conserved in patients, with either Stage II or Stage III colorectal cancer, who did not have cancer recurrence. Therefore, such tumors represent useful reference samples for miRNA profile comparisons in colorectal cancer patient prognosis.

### Example 4

### QRT-PCR Verification of Differentially Expressed Micro-RNAs in Tumors from Colorectal Cancer Patients with Cancer Recurrence

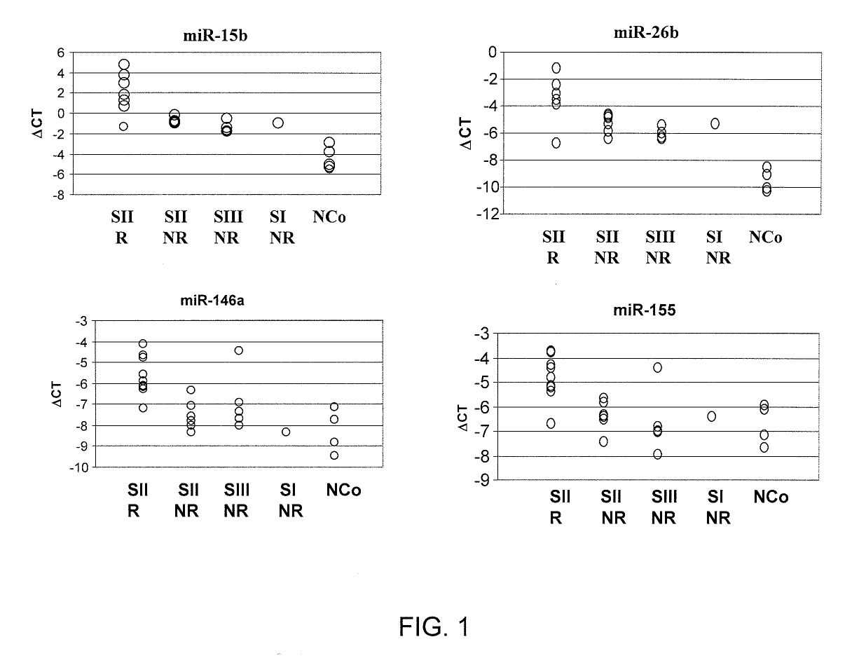

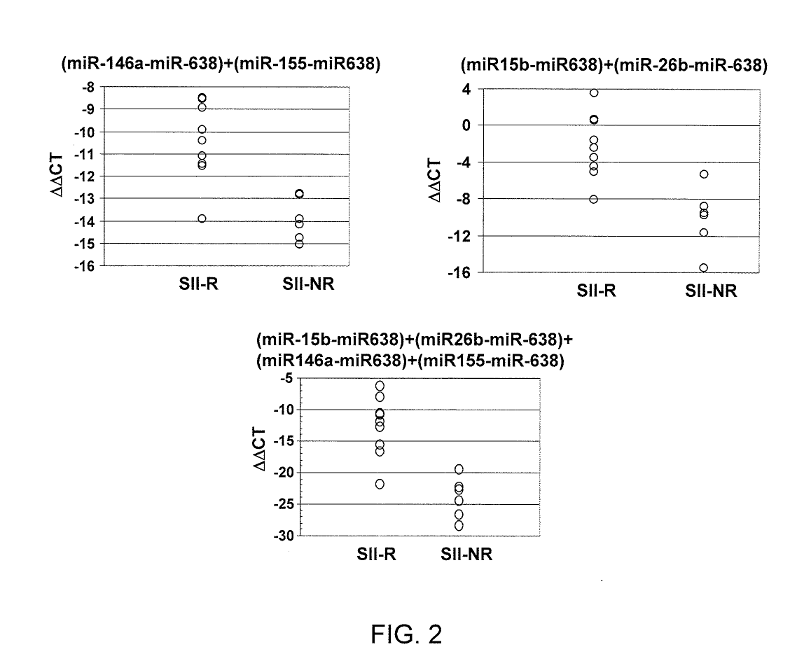

The inventors evaluated the differential expression of selected miRNAs in tumors from colorectal cancer patients who had cancer recurrence or who had no recurrence and in normal colon samples (FirstChoice® Human Colon Total RNA, cat. no. AM7986; Ambion Inc., Austin, Tex., USA) by qRT-PCR. qRT-PCR reactions were performed using TaqMan® MicroRNA Assays (Applied Biosystems; Foster City, Calif., USA) specific for four miRNAs (hsa-miR-15b, -26b, -146a, and -155) that were shown by microarray analysis to be expressed at lower levels in tumors from patients (Stage II) with colon cancer recurrence than in tumors from patients (Stage I, II, or III) with no cancer recurrence. Reverse transcription was performed in a 10 μl reaction volume containing 15 ng of total RNA, and reactions were incubated in an Applied Biosystems 9800 Fast Thermal Cycler (Applied Biosystems) in a 96-well plate for 30 min at 16° C., then 30 min at 42° C., and finally 5 min at 85° C. For PCR, 2 μl of the RT reactions were added to PCR reactions in a final volume of 15 Real-time PCR was performed using the 7900HT Fast Real-Time PCR system (Applied Biosystems). miR-638, whose expression level showed little variation among tumors from all cancer stages irrespective of recurrence status, was used for normalization. Any miRNA or other nucleic acid whose expression level shows no or little variation among tumors from all cancers, irrespective of recurrent status can be used for normalization. When compared to normal colon tissue and to cancers from non-recurrent stages I, II, and III, cancers from recurrent stage II showed down regulation of miR-15b, miR-26b, miR146a and miR155 (FIG. 1). Using data from combinations of microRNAs shown in FIG. 1 enhances separation between Stage II colorectal cancer patients who had recurrence (SII R) and Stage II patients who had no recurrence (SII NR) within five years of initial surgery of primary tumor (FIG. 2). Data from combinations of miRs increases specificity and sensitivity in predicting the likelihood of colorectal cancer recurrence over data from a single miRNA.

These data demonstrate that qRT-PCR quantification of selected miRNAs is an effective method for quantifying miRNA expression when predicting the likelihood of colon cancer recurrence.

### Example 5

### Analysis of Stage II Colorectal Cancer Tumors and Identification of MicroRNA Biomarkers for Cancer Recurrence

The inventors used microarray quantification to identify miRNAs that are useful for distinguishing patients with Stage II colon cancer and having a high risk of cancer recurrence from patients with Stage II colon cancer and having no recurrence within a five-year period. Tumor samples are described in Table 7 and Table 8. miRNA isolation and purification were performed as described in Example 1.

TABLE 8
Histopathological data and patient information for colorectal cancer tissue samples.
Samples 7 and 7a are separate samples of colon tissue taken from the same patient. R, recurrent;
NR, non-recurrent; RLQ, right lower liver quadrant; NA, not available;-, not applicable; LN,
lymph nodes.

| | Patient | | Clinical Stage | TNM Status | Grade | | Cancer- | Time to | Distant |
|---|---|---|---|---|---|---|---|---|---|
| | Information | | (Greene et | (Greene et | (Crissman | | Recurrence | Recurrence | Metastasis |
| Sample # | Sex | Age | al., 2002) | al., 2002) | et al., 1989) | Tissue | Status | (months) | Site |
| 1 | F | 50 | IIA | T3N0M0 | NA | cecum | NR | — | — |
| 2 | M | 80 | IIA | T3N0M0 | 2 | colon | NR | — | — |
| 3 | M | 47 | IIA | T3N0M0 | NA | colon | NR | — | — |
| 4 | F | 70 | I | T2N0M0 | 2 | colon | NR | — | — |
| 5 | F | 86 | IIA | T3N0M0 | 2 | colon | NR | — | — |
| 6 | F | 75 | IIA | T3N0M0 | NA | cecum | NR | — | — |
| 7 | F | 77 | IIA | T3N0M0 | 2 | colon | R | 26 | lung |
| 7a | F | 77 | IIA | T3N0M0 | 2 | colon | R | 26 | lung |
| 8 | F | 69 | IIA | T3N0M0 | 2 | colon | R | 25 | lung liver |
| 9 | M | 73 | IIA | T3N0M0 | 2 | colon | R | 36 | lung |
| 10 | M | 80 | IIA | T3N0M0 | 3 | colon | R | 36 | liver |
| 11 | M | 43 | IIA | T3N0M0 | NA | colon | R | 33 | liver |
| 12 | M | 72 | IIA | T3N0M0 | NA | colon | R | 9 | liver |
| 13 | F | 66 | IIA | T3N0M0 | 2 | colon | R | 49 | uterus |
| 14 | F | 85 | IIA | T3N0M0 | 3 | cecum | R | 39,44,44 | liver, colon, LN |
| 15 | F | 77 | IIB | T4N0M0 | 2 | colon | R | 1,5 | RLQ mass, bone |

TABLE 9
Histopathological data and patient information for colorectal cancer tissue
Samples 16 and 16a are separate samples of colon tissue taken from the same patient. R,
recurrent; NR, non-recurrent; RLQ, right lower liver quadrant; NA, not available;-, not
applicable; LN, lymph nodes.

| | | | Clinical Stage | TNM Status | Grade | | Cancer- | Time to | Distant |
|---|---|---|---|---|---|---|---|---|---|
| | Patient Information | | (Greene et | (Greene et | (Crissman | | Recurrence | Recurrence | Metastasis |
| Sample # | Sex | Age | al., 2002) | al., 2002) | et al., 1989) | Tissue | Status | (months) | Site |
| 1 | F | 36 | IIA | T3N0M0 | NA | colon | NR | — | — |
| 2 | F | 53 | IIA | T3N0M0 | X | cecum | NR | — | — |
| 3 | M | 60 | IIA | T3N0M0 | 2 | colon | NR | — | — |
| 4 | M | 66 | IIA | T3N0M0 | 3 | cecum | NR | — | — |
| 5 | M | 68 | IIA | T3N0M0 | 1 | colon | NR | — | — |
| 6 | M | 71 | IIA | T3N0M0 | 2 | colon | NR | — | — |
| 7 | M | 72 | IIA | T3N0M0 | 2 | colon | NR | — | — |
| 8 | M | 75 | IIA | T3N0MX | NA | colon | NR | — | — |
| 9 | M | 76 | IIA | T3N0M0 | 1 | colon | NR | — | — |
| 10 | M | 78 | IIA | T3N0M0 | 2 | colon | NR | — | — |
| 11 | F | 80 | IIA | T3N0M0 | X | colon | NR | — | — |
| 12 | F | 84 | IIA | T3N0M0 | 2 | colon | NR | — | — |
| 13 | F | 85 | IIA | T3N0M0 | 3 | colon | NR | — | — |
| 14 | F | 90 | IIA | T3N0M0 | 2 | colon | NR | — | — |
| 15 | F | 60 | IIA | T3N0M0 | 1 | colon | R | 24 | ovary |
| 16 | F | 77 | IIA | T3N0M0 | 2 | colon | R | 26 | lung |
| 16a | F | 77 | IIA | T3N0M0 | 2 | colon | R | 26 | lung |

Samples were evaluated using the Agilent Human miRNA Microarray (V2) (Agilent Technologies, Inc.; Santa Clara, Calif. USA), which contains probes for 723 human and 76 human viral microRNAs from the miRBase database V.10.1 (Griffiths-Jones et al., 2006). Array hybridization, washing, staining, imaging, and signal extraction were performed according to Agilent's recommended procedures, as explained below.

miRNA Array Expression Analysis:

Samples for miRNA profiling studies were processed by Asuragen Services (Asuragen, Inc.; Austin, Tex., USA). Total RNA from each sample (100 ng) was dephosphorylated and a pCp-Cy3 labeling molecule was ligated to the 3′ end of the RNA molecules, following the manufacturer's recommendations (Agilent Technologies, Inc. Santa Clara, USA). The labeled RNA was purified using a Bio-Spin P-6 column (Bio-Rad Laboratories Inc.; Hercules, Calif., USA).

miRNA Array Signal Processing:

The signal processing implemented for the Agilent miRNA array is a multi-step process involving probe specific signal detection calls, background correction, and global normalization. For each probe, the contribution of signal due to background was estimated and removed by the Agilent Feature Extraction software as part of the data file output. Similarly, detection calls were based on the Agilent Feature Extraction software. Arrays within a specific analysis experiment were normalized together according to the VSN method described elsewhere (Huber et al., 2002).

Background Estimate and Correction and Probe Detection:

Three types of data are provided to evaluate each hybridization. The “Total Gene Signal” is the total probe signal multiplied by the number of probes per gene and is calculated after the background effects have been accounted for. The “Total Gene Error” is the square root of the square of the total probe error multiplied by the number of probes per gene. The “Total Probe Error” is the robust average for each replicated probe multiplied by the total number of probe replicates. The “Detection Call” is a binary number that indicates if the gene was detected on the miRNA microarray. Probes detected at least once across all samples in the experiment were considered for statistical analysis.

Global Normalization:

The inventors have found that the Variance Stabilization Normalization (VSN) algorithm provides an ideal balance of accuracy and precision while optimizing sensitivity and specificity of signal. One advantage of VSN, is that it accommodates negative values by using the generalized log₂ transformation.

Generalized log₂ Transformed:

The post-normalized data scale is reported as generalized log₂ data. The distribution of microarray data is typically log normal (i.e., it tends to follow a normal distribution pattern after log transformation). A normal distributed data is amendable to classical statistical treatments, including t-tests and one-way or two-way ANOVA.

For statistical hypothesis testing, a two-sample t-test, with assumption of equal variance, was applied. This test is used to define which probes are considered to be significantly differentially expressed, or “significant”, based on false discovery rate set at 0.05.

Using the specimens in Table 7, miRNA expression levels in tissue samples from patients with recurrent cancer were compared with miRNA expression levels in tissue samples from patients with non-recurring cancer. The inventors identified differentially expressed miRNAs, with the indicated ttest p-values (Table 9), that could distinguish patients with recurrent, Stage II colon caner from patients with non-recurrent Stage II colon cancer.

TABLE 10
MicroRNAs Differentially Expressed Between Tumor Samples
from Patients with Stage II Colorectal Cancer (non-recurrent
vs recurrent). Mean, mean of normalized array data for ten
tumor samples from patients with cancer recurrence (R) and
for six tumor samples from patients without cancer recurrence
(NR). Log2Diff, difference in VSN-transformed expression
between tumor samples from patients with non-recurrent and
recurrent cancer; FC, Fold Change.

| | Stage II | Stage II | | | |
|---|---|---|---|---|---|
| | NR | R | R vs NR | | |
| miRNA | Mean | Mean | Log2Diff | FC | ttest |
| hsa-miR-23a* | 2.45 | 0.11 | −2.34 | 5.05 | 1.35E−03 |
| hsa-miR-501-5p | 4.75 | 2.44 | −2.31 | 4.94 | 4.07E−05 |
| hsa-miR-224 | −0.08 | 2.19 | 2.27 | 4.81 | 1.81E−03 |
| hsa-miR-551b* | 0.96 | −1.29 | −2.24 | 4.73 | 2.22E−03 |
| hsa-miR-15b | 4.02 | 6.25 | 2.23 | 4.69 | 9.82E−05 |
| hsa-miR-500 | 5.03 | 2.83 | −2.20 | 4.60 | 6.82E−05 |
| hsa-let-7a | 5.88 | 8.05 | 2.17 | 4.50 | 2.85E−04 |
| hsa-miR-192* | 0.21 | 2.29 | 2.08 | 4.23 | 1.07E−04 |
| hsa-miR-28-3p | 0.81 | −1.20 | −2.01 | 4.02 | 3.21E−05 |
| hsa-let-7f | 4.53 | 6.53 | 2.00 | 4.01 | 5.31E−04 |
| hsa-let-7c* | 1.02 | −0.93 | −1.96 | 3.88 | 1.01E−04 |
| hsa-miR-17 | 3.77 | 5.70 | 1.93 | 3.82 | 7.84E−05 |
| hsa-miR-200b | 5.78 | 7.67 | 1.89 | 3.70 | 6.39E−04 |
| hsa-let-7d | 3.91 | 5.78 | 1.87 | 3.65 | 5.10E−05 |
| hsa-miR-616 | −0.31 | −2.17 | −1.85 | 3.61 | 4.26E−04 |
| hsa-miR-30b | 1.83 | 3.66 | 1.83 | 3.55 | 2.41E−04 |
| hsa-miR-20a | 5.18 | 7.01 | 1.83 | 3.54 | 7.59E−04 |
| hsa-miR-425 | 1.99 | 3.79 | 1.79 | 3.46 | 3.81E−04 |
| hsa-miR-886-3p | 4.37 | 6.15 | 1.77 | 3.42 | 9.97E−04 |
| hsa-miR-125b | 3.24 | 5.01 | 1.77 | 3.41 | 1.37E−02 |
| hsa-miR-20b | 1.41 | 3.15 | 1.74 | 3.35 | 1.45E−04 |
| hsa-miR-135b* | −0.81 | −2.55 | −1.74 | 3.34 | 2.77E−03 |
| hsa-miR-892b | 4.71 | 2.97 | −1.74 | 3.34 | 6.25E−04 |
| hsa-let-7g | 4.44 | 6.14 | 1.70 | 3.24 | 2.04E−03 |
| hsa-miR-508-5p | 0.71 | −0.98 | −1.69 | 3.22 | 1.58E−02 |
| hsa-miR-629 | −0.13 | −1.81 | −1.68 | 3.20 | 3.07E−03 |
| hsa-miR-93 | 2.60 | 4.26 | 1.65 | 3.14 | 3.17E−04 |
| hsa-miR-199b-3p | 3.98 | 5.60 | 1.63 | 3.09 | 1.05E−02 |
| hsa-miR-183* | 0.63 | −0.98 | −1.61 | 3.05 | 2.20E−03 |
| hsa-miR-331-5p | −0.38 | −1.98 | −1.59 | 3.02 | 5.81E−04 |
| hsa-miR-596 | 2.52 | 0.93 | −1.59 | 3.01 | 2.57E−03 |
| hsa-miR-103 | 3.91 | 5.50 | 1.58 | 3.00 | 1.82E−03 |
| hsa-miR-92a | 4.05 | 5.63 | 1.58 | 2.99 | 1.52E−03 |
| hsa-let-7b | 7.97 | 9.54 | 1.57 | 2.96 | 1.75E−03 |
| hsa-miR-509-3-5p | 1.84 | 0.28 | −1.56 | 2.94 | 8.49E−03 |
| hsa-let-7c | 4.04 | 5.60 | 1.56 | 2.94 | 1.23E−02 |
| hsa-miR-26b | 2.10 | 3.65 | 1.55 | 2.92 | 1.52E−02 |
| hsa-miR-552 | 0.45 | 1.99 | 1.54 | 2.91 | 2.82E−02 |
| hsa-miR-210 | 3.93 | 5.46 | 1.53 | 2.89 | 7.16E−03 |
| hsa-miR-768-3p | 5.16 | 6.69 | 1.53 | 2.88 | 1.67E−03 |
| hsa-miR-23b | 4.52 | 6.04 | 1.52 | 2.87 | 5.72E−03 |
| hsa-miR-107 | 3.52 | 5.04 | 1.52 | 2.87 | 2.14E−03 |
| hsa-miR-19b | 3.89 | 5.40 | 1.51 | 2.85 | 1.14E−03 |
| hsa-miR-192 | 6.51 | 8.02 | 1.51 | 2.84 | 9.73E−04 |
| hsa-miR-675 | −0.98 | −2.49 | −1.50 | 2.83 | 6.42E−03 |
| hsa-miR-890 | −0.67 | −2.17 | −1.50 | 2.83 | 1.45E−03 |
| hsa-miR-203 | 0.05 | 1.55 | 1.49 | 2.81 | 4.08E−03 |
| hsa-let-7e | 4.27 | 5.75 | 1.48 | 2.79 | 1.12E−02 |
| hsa-miR-145 | 4.36 | 5.84 | 1.48 | 2.78 | 4.02E−02 |
| hsa-miR-451 | 3.45 | 4.93 | 1.48 | 2.78 | 1.84E−02 |
| hsa-miR-95 | −0.53 | 0.94 | 1.47 | 2.77 | 2.09E−03 |
| hsa-miR-151-5p | 3.00 | 4.46 | 1.46 | 2.75 | 1.17E−03 |
| hsa-miR-24 | 5.81 | 7.27 | 1.45 | 2.74 | 1.20E−03 |
| hsa-miR-376c | −1.30 | 0.15 | 1.45 | 2.74 | 1.34E−02 |
| hsa-miR-374a | −0.74 | 0.69 | 1.43 | 2.70 | 4.11E−03 |
| hsa-miR-200c | 5.47 | 6.90 | 1.43 | 2.69 | 7.63E−03 |
| hsa-miR-194 | 4.55 | 5.98 | 1.42 | 2.68 | 2.15E−03 |
| hsa-miR-23a | 5.16 | 6.58 | 1.41 | 2.67 | 4.34E−03 |
| hsa-miR-185 | −0.22 | 1.18 | 1.40 | 2.64 | 2.99E−02 |
| hsa-miR-455-3p | 0.05 | 1.45 | 1.40 | 2.63 | 6.13E−03 |
| hsa-miR-202 | 3.55 | 2.15 | −1.39 | 2.63 | 4.53E−03 |
| hsa-miR-302c* | 0.83 | −0.56 | −1.39 | 2.62 | 5.60E−03 |
| hsa-miR-19a | 0.68 | 2.06 | 1.38 | 2.60 | 6.82E−03 |
| hsa-miR-877 | 4.63 | 3.26 | −1.38 | 2.60 | 3.04E−03 |
| hsa-miR-139-3p | 3.49 | 2.12 | −1.37 | 2.59 | 1.32E−03 |
| hsa-miR-375 | 1.37 | 2.74 | 1.37 | 2.58 | 9.19E−03 |
| hsa-miR-493 | 1.41 | 0.04 | −1.36 | 2.57 | 1.65E−02 |
| hsa-miR-374b | −0.27 | 1.08 | 1.35 | 2.56 | 9.52E−03 |
| hsa-miR-100 | 3.28 | 4.63 | 1.35 | 2.55 | 2.31E−02 |
| hsa-miR-27b | 4.29 | 5.64 | 1.35 | 2.55 | 7.86E−03 |
| hsa-miR-25 | 3.76 | 5.08 | 1.32 | 2.49 | 3.11E−03 |
| hsa-miR-18a | −0.93 | 0.37 | 1.31 | 2.47 | 3.14E−02 |
| hsa-miR-200a | 2.82 | 4.12 | 1.30 | 2.47 | 2.55E−02 |
| hsa-miR-206 | 0.46 | −0.83 | −1.29 | 2.45 | 4.80E−02 |
| hsa-miR-10a | 4.53 | 5.82 | 1.29 | 2.45 | 4.73E−02 |
| hsa-miR-125b-1* | 2.84 | 1.56 | −1.28 | 2.43 | 2.14E−03 |
| hsa-miR-632 | 1.73 | 0.46 | −1.27 | 2.41 | 4.64E−05 |
| hsa-miR-16 | 5.94 | 7.20 | 1.26 | 2.39 | 3.22E−03 |
| hsa-miR-125a-5p | 2.03 | 3.28 | 1.25 | 2.38 | 7.91E−03 |
| hsa-miR-125b-2* | 2.13 | 0.89 | −1.24 | 2.36 | 2.43E−04 |
| hsa-miR-183 | 1.35 | 2.56 | 1.21 | 2.31 | 6.67E−03 |
| hsa-miR-424 | 0.83 | 2.03 | 1.20 | 2.30 | 5.97E−04 |
| hsa-miR-221 | 0.92 | 2.12 | 1.20 | 2.29 | 1.81E−02 |
| hsa-miR-214 | 2.88 | 4.07 | 1.19 | 2.29 | 1.47E−02 |
| hsa-miR-26a | 3.97 | 5.15 | 1.18 | 2.27 | 2.47E−02 |
| hsa-miR-429 | 2.46 | 3.63 | 1.17 | 2.25 | 3.99E−02 |
| hsa-miR-193b | 2.03 | 3.19 | 1.16 | 2.24 | 1.54E−03 |
| hsa-miR-361-5p | 2.64 | 3.80 | 1.16 | 2.23 | 1.70E−02 |
| hsa-miR-363 | 0.19 | 1.34 | 1.15 | 2.22 | 9.94E−04 |
| hsa-miR-658 | 0.50 | −0.65 | −1.15 | 2.22 | 2.55E−03 |
| hsa-miR-148a | 2.33 | 3.43 | 1.10 | 2.14 | 4.63E−02 |
| hsa-miR-512-3p | 3.75 | 2.65 | −1.10 | 2.14 | 1.11E−03 |
| hsa-miR-124 | 0.64 | −0.43 | −1.07 | 2.10 | 3.32E−02 |
| hsa-miR-10b | 2.89 | 3.96 | 1.07 | 2.10 | 4.85E−02 |
| hsa-miR-199b-5p | 1.32 | 2.39 | 1.07 | 2.10 | 3.09E−02 |
| hsa-miR-194* | 1.01 | 2.07 | 1.06 | 2.08 | 8.65E−05 |
| hsa-miR-625 | 0.69 | 1.74 | 1.06 | 2.08 | 5.14E−03 |
| hsa-miR-148b | −0.90 | 0.16 | 1.05 | 2.08 | 8.12E−03 |
| hsa-miR-140-5p | 1.30 | 2.34 | 1.05 | 2.07 | 1.84E−02 |
| hsa-miR-29a | 6.30 | 7.33 | 1.03 | 2.04 | 1.64E−02 |
| hsa-miR-128 | −0.39 | 0.63 | 1.02 | 2.03 | 1.08E−02 |
| hsa-miR-30c | 2.19 | 3.21 | 1.02 | 2.03 | 5.31E−03 |
| hsa-miR-34a | 5.24 | 6.26 | 1.02 | 2.03 | 3.20E−02 |
| hsa-miR-605 | 2.35 | 1.34 | −1.01 | 2.02 | 4.02E−03 |
| hsa-miR-708 | 2.34 | 1.34 | −1.00 | 2.00 | 7.76E−04 |
| hsa-miR-760 | 3.70 | 2.70 | −1.00 | 1.99 | 9.60E−03 |
| hsa-miR-518d-5p | 0.64 | −0.36 | −0.99 | 1.99 | 7.23E−03 |
| hsa-miR-491-5p | 1.09 | 0.10 | −0.99 | 1.99 | 4.00E−04 |
| hsa-miR-28-5p | 3.24 | 4.23 | 0.99 | 1.98 | 1.13E−02 |
| hsa-miR-650 | 2.69 | 1.71 | −0.98 | 1.97 | 4.75E−02 |
| hsa-miR-877* | 3.06 | 4.04 | 0.98 | 1.97 | 7.92E−06 |
| hsa-miR-610 | 3.04 | 2.06 | −0.98 | 1.97 | 1.42E−03 |
| hsa-miR-629* | 3.60 | 2.63 | −0.97 | 1.96 | 2.54E−02 |
| hsa-miR-181c* | 0.81 | −0.15 | −0.96 | 1.95 | 3.28E−03 |
| hsa-miR-215 | 4.05 | 4.99 | 0.95 | 1.93 | 7.09E−03 |
| hsa-miR-186* | 0.81 | −0.13 | −0.93 | 1.91 | 5.13E−03 |
| hsa-miR-200b* | 1.89 | 2.81 | 0.92 | 1.89 | 4.40E−03 |
| hsa-miR-1226* | 5.27 | 4.36 | −0.91 | 1.88 | 2.20E−03 |
| hsa-miR-432 | 2.05 | 1.15 | −0.89 | 1.86 | 2.27E−03 |
| hsa-miR-601 | 4.78 | 3.90 | −0.88 | 1.84 | 1.88E−02 |
| hsa-miR-149* | 3.66 | 2.78 | −0.88 | 1.84 | 2.95E−03 |
| hsa-miR-126 | 3.43 | 4.30 | 0.87 | 1.83 | 3.38E−02 |
| hsa-miR-409-3p | 1.35 | 2.21 | 0.86 | 1.82 | 2.32E−05 |
| hsa-miR-129-5p | 2.04 | 1.18 | −0.86 | 1.81 | 4.65E−02 |
| hsa-miR-34b* | −0.03 | 0.82 | 0.85 | 1.80 | 3.58E−03 |
| hsa-miR-331-3p | 4.07 | 4.90 | 0.82 | 1.77 | 1.63E−02 |
| hsa-miR-29b-1* | −0.14 | 0.68 | 0.82 | 1.77 | 2.42E−02 |
| hsa-miR-31* | −0.63 | 0.19 | 0.82 | 1.76 | 2.26E−02 |
| hsa-miR-542-5p | 2.98 | 2.17 | −0.81 | 1.76 | 5.08E−02 |
| hsa-miR-132 | 0.67 | 1.48 | 0.81 | 1.75 | 3.31E−02 |
| hsa-miR-133b | 2.46 | 3.24 | 0.78 | 1.72 | 1.81E−02 |
| hsa-miR-151-3p | 2.11 | 2.89 | 0.78 | 1.72 | 4.41E−02 |
| hsa-miR-513a-3p | 1.25 | 0.48 | −0.77 | 1.71 | 1.41E−02 |
| hsa-miR-486-5p | 3.77 | 3.00 | −0.77 | 1.70 | 3.25E−03 |
| hsa-miR-135b | 0.30 | 1.03 | 0.73 | 1.66 | 2.91E−02 |
| hsa-miR-138-2* | 2.17 | 1.44 | −0.73 | 1.66 | 3.13E−02 |
| hsa-miR-96 | 0.98 | 1.71 | 0.73 | 1.66 | 2.64E−02 |
| hsa-miR-373 | 0.66 | 0.00 | −0.66 | 1.58 | 5.03E−02 |
| hsa-miR-584 | 2.58 | 1.93 | −0.65 | 1.57 | 3.23E−02 |
| hsa-miR-526b* | 0.81 | 0.18 | −0.63 | 1.55 | 3.41E−02 |
| hsa-miR-769-3p | 3.16 | 2.56 | −0.60 | 1.51 | 2.02E−02 |
| hsa-miR-296-5p | 4.36 | 4.95 | 0.59 | 1.50 | 1.34E−03 |
| hsa-miR-30d | 4.13 | 4.71 | 0.58 | 1.50 | 4.43E−02 |

Similarly, in the set of specimens described in Table 8, miRNA expression levels in tissue samples from patients with recurrent cancer were compared with miRNA expression levels in tissue samples from patients with non-recurring cancer. With this tissue set, the inventors identified miRNAs listed in Table 10 that were differentially expressed between patients with recurrent and non-recurrent colon cancers.

TABLE 11
MicroRNAs Differentially Expressed Between Tumor Samples
from Patients with Stage II Colorectal Cancer (non-recurrent
vs recurrent). Mean, mean of normalized array data for 14
tumor samples from patients with cancer recurrence (R) and
for three tumor samples from patients without cancer recurrence
(NR). Log2Diff, difference in VSN-transformed expression
between tumor samples from patients with non-recurrent and
recurrent cancer; FC, Fold Change.

| | Stage II | | | | |
|---|---|---|---|---|---|
| | NR | Stage II R | NR vs R | | |
| miRNA | Mean | Mean | Log2Diff | FC | ttest |
| hsa-miR-146a | 2.37 | −0.67 | 3.04 | 8.20 | 1.50E−03 |
| hsa-miR-146b-5p | 1.75 | −1.06 | 2.81 | 7.02 | 9.74E−04 |
| hsa-miR-223 | 6.16 | 3.44 | 2.72 | 6.59 | 2.82E−03 |
| hsa-miR-15b | 6.36 | 3.73 | 2.64 | 6.22 | 6.07E−04 |
| hsa-miR-185 | 1.36 | −1.00 | 2.36 | 5.14 | 8.65E−03 |
| hsa-let-7a | 8.11 | 5.75 | 2.36 | 5.12 | 5.18E−03 |
| hsa-miR-103 | 5.66 | 3.31 | 2.34 | 5.08 | 2.28E−03 |
| hsa-let-7e | 6.24 | 3.94 | 2.31 | 4.95 | 5.97E−03 |
| hsa-let-7f | 6.53 | 4.26 | 2.27 | 4.83 | 6.88E−03 |
| hsa-miR-107 | 5.25 | 3.08 | 2.17 | 4.50 | 1.46E−03 |
| hsa-let-7g | 6.09 | 3.95 | 2.15 | 4.42 | 7.46E−03 |
| hsa-miR-768-3p | 6.68 | 4.56 | 2.13 | 4.37 | 8.08E−05 |
| hsa-miR-374a | 0.60 | −1.52 | 2.12 | 4.34 | 1.91E−03 |
| hsa-miR-148a | 3.07 | 0.96 | 2.10 | 4.30 | 9.62E−03 |
| hsa-miR-501-5p | 2.33 | 4.43 | −2.09 | 4.27 | 3.15E−04 |
| hsa-miR-183 | 2.95 | 0.87 | 2.08 | 4.24 | 7.90E−03 |
| hsa-miR-140-3p | 2.90 | 0.81 | 2.08 | 4.23 | 6.20E−04 |
| hsa-miR-23a | 6.70 | 4.67 | 2.03 | 4.09 | 8.05E−03 |
| hsa-miR-125b | 4.95 | 2.96 | 1.99 | 3.98 | 1.84E−02 |
| hsa-miR-19a | 1.64 | −0.34 | 1.98 | 3.93 | 5.17E−03 |
| hsa-miR-210 | 5.37 | 3.41 | 1.96 | 3.90 | 2.47E−03 |
| hsa-miR-374b | 1.17 | −0.80 | 1.96 | 3.90 | 4.27E−03 |
| hsa-miR-34a | 6.16 | 4.21 | 1.95 | 3.86 | 1.18E−02 |
| hsa-miR-151-5p | 4.43 | 2.49 | 1.94 | 3.84 | 7.46E−03 |
| hsa-miR-150 | 4.41 | 2.48 | 1.93 | 3.82 | 2.08E−03 |
| hsa-miR-16 | 7.16 | 5.23 | 1.93 | 3.81 | 3.92E−03 |
| hsa-miR-93 | 4.25 | 2.32 | 1.93 | 3.81 | 9.12E−03 |
| hsa-let-7d | 5.83 | 3.91 | 1.92 | 3.80 | 6.04E−03 |
| hsa-miR-199b-3p | 5.58 | 3.69 | 1.89 | 3.70 | 1.74E−02 |
| hsa-miR-24 | 7.37 | 5.49 | 1.88 | 3.68 | 6.11E−03 |
| hsa-miR-17 | 5.52 | 3.64 | 1.88 | 3.68 | 6.75E−03 |
| hsa-miR-95 | 0.93 | −0.91 | 1.84 | 3.59 | 1.04E−02 |
| hsa-miR-10b | 4.10 | 2.27 | 1.83 | 3.56 | 1.48E−02 |
| hsa-miR-10a | 5.79 | 3.98 | 1.81 | 3.51 | 2.33E−02 |
| hsa-let-7c | 5.62 | 3.81 | 1.81 | 3.50 | 1.69E−02 |
| hsa-miR-25 | 5.04 | 3.28 | 1.76 | 3.39 | 1.38E−02 |
| hsa-miR-125a-5p | 3.70 | 1.97 | 1.73 | 3.32 | 1.86E−02 |
| hsa-miR-361-5p | 3.57 | 1.87 | 1.70 | 3.24 | 1.15E−02 |
| hsa-miR-140-5p | 2.29 | 0.60 | 1.69 | 3.23 | 2.16E−02 |
| hsa-miR-100 | 4.64 | 2.97 | 1.68 | 3.19 | 1.42E−02 |
| hsa-miR-214 | 4.09 | 2.42 | 1.67 | 3.19 | 2.67E−02 |
| hsa-miR-135b* | −2.64 | −0.97 | −1.67 | 3.18 | 5.20E−03 |
| hsa-miR-500 | 2.72 | 4.38 | −1.66 | 3.16 | 1.03E−02 |
| hsa-miR-99b | 2.40 | 0.74 | 1.66 | 3.16 | 1.58E−02 |
| hsa-miR-126 | 4.26 | 2.63 | 1.63 | 3.10 | 1.49E−02 |
| hsa-miR-29a | 7.07 | 5.44 | 1.62 | 3.08 | 2.27E−02 |
| hsa-miR-30b | 3.48 | 1.86 | 1.62 | 3.08 | 7.07E−03 |
| hsa-let-7b | 9.60 | 7.98 | 1.62 | 3.08 | 1.41E−02 |
| hsa-miR-26b | 3.52 | 1.90 | 1.62 | 3.07 | 3.64E−02 |
| hsa-miR-23b | 5.89 | 4.29 | 1.60 | 3.04 | 1.31E−02 |
| hsa-miR-20a | 6.73 | 5.13 | 1.59 | 3.02 | 3.34E−02 |
| hsa-miR-892b | 2.93 | 4.52 | −1.59 | 3.01 | 4.61E−03 |
| hsa-miR-532-5p | 2.06 | 0.49 | 1.57 | 2.97 | 3.84E−02 |
| hsa-miR-92a | 5.35 | 3.79 | 1.56 | 2.96 | 2.52E−02 |
| hsa-miR-455-3p | 1.68 | 0.12 | 1.56 | 2.95 | 1.03E−02 |
| hsa-miR-675 | −2.44 | −0.89 | −1.55 | 2.94 | 4.08E−03 |
| hsa-miR-200b | 7.35 | 5.81 | 1.54 | 2.91 | 4.68E−02 |
| hsa-let-7i | 7.17 | 5.64 | 1.53 | 2.88 | 2.72E−02 |
| hsa-miR-26a | 4.98 | 3.47 | 1.51 | 2.85 | 2.53E−02 |
| hsa-miR-886-3p | 5.61 | 4.11 | 1.51 | 2.85 | 6.61E−03 |
| hsa-miR-181a | 4.28 | 2.78 | 1.49 | 2.81 | 1.12E−02 |
| hsa-miR-342-3p | 4.18 | 2.73 | 1.45 | 2.74 | 3.75E−02 |
| hsa-miR-132 | 1.59 | 0.17 | 1.43 | 2.69 | 2.78E−02 |
| hsa-miR-27b | 5.43 | 4.01 | 1.42 | 2.68 | 3.87E−02 |
| hsa-miR-425 | 3.67 | 2.26 | 1.40 | 2.64 | 8.31E−03 |
| hsa-miR-632 | 0.42 | 1.80 | −1.38 | 2.60 | 2.78E−05 |
| hsa-miR-424 | 1.87 | 0.50 | 1.37 | 2.59 | 7.89E−03 |
| hsa-miR-324-5p | 3.01 | 1.66 | 1.35 | 2.55 | 1.67E−02 |
| hsa-miR-424* | 2.33 | 1.02 | 1.31 | 2.48 | 6.94E−03 |
| hsa-miR-21* | 3.58 | 2.27 | 1.31 | 2.48 | 2.68E−02 |
| hsa-miR-331-3p | 5.00 | 3.69 | 1.31 | 2.48 | 2.67E−02 |
| hsa-let-7c* | −0.66 | 0.64 | −1.30 | 2.47 | 1.18E−02 |
| hsa-miR-29b-1* | 0.67 | −0.63 | 1.30 | 2.46 | 2.95E−02 |
| hsa-miR-193b | 3.07 | 1.77 | 1.30 | 2.46 | 1.64E−02 |
| hsa-miR-615-3p | −0.02 | 1.28 | −1.30 | 2.46 | 3.82E−03 |
| hsa-miR-200b* | 2.49 | 1.20 | 1.29 | 2.45 | 3.74E−02 |
| hsa-miR-409-3p | 2.32 | 1.06 | 1.27 | 2.41 | 2.27E−04 |
| hsa-miR-491-5p | −0.29 | 0.98 | −1.27 | 2.41 | 1.99E−02 |
| hsa-miR-801 | 6.53 | 5.30 | 1.22 | 2.34 | 5.20E−03 |
| hsa-miR-1224-3p | 0.13 | 1.36 | −1.22 | 2.33 | 3.88E−03 |
| hsa-miR-20b | 2.88 | 1.67 | 1.21 | 2.31 | 1.15E−02 |
| hsa-miR-596 | 0.97 | 2.14 | −1.17 | 2.25 | 2.77E−02 |
| hsa-miR-151-3p | 2.82 | 1.65 | 1.16 | 2.24 | 4.14E−02 |
| hsa-miR-125b-2* | 0.96 | 2.11 | −1.15 | 2.22 | 1.33E−04 |
| hsa-miR-518d-5p | −0.72 | 0.40 | −1.12 | 2.17 | 2.55E−02 |
| hsa-miR-613 | 0.24 | 1.35 | −1.11 | 2.16 | 1.98E−03 |
| hsa-miR-485-3p | 0.54 | 1.64 | −1.10 | 2.14 | 8.10E−03 |
| hsa-miR-28-5p | 4.07 | 2.98 | 1.10 | 2.14 | 4.82E−02 |
| hsa-miR-768-5p | 6.34 | 5.24 | 1.10 | 2.14 | 2.97E−04 |
| hsa-let-7d* | 0.78 | 1.87 | −1.10 | 2.14 | 1.32E−02 |
| hsa-miR-885-5p | 0.64 | 1.74 | −1.10 | 2.14 | 2.39E−02 |
| hsa-miR-519d | 0.42 | 1.50 | −1.09 | 2.12 | 2.23E−03 |
| hsa-miR-127-3p | 1.96 | 0.88 | 1.08 | 2.11 | 3.85E−02 |
| hsa-miR-769-5p | 1.53 | 0.46 | 1.07 | 2.10 | 3.99E−02 |
| hsa-miR-30d | 4.71 | 3.65 | 1.07 | 2.09 | 2.83E−02 |
| hsa-miR-328 | 1.43 | 2.48 | −1.05 | 2.07 | 2.38E−02 |
| hsa-miR-1228 | 5.90 | 4.86 | 1.04 | 2.05 | 1.88E−03 |
| hsa-miR-501-3p | 0.66 | −0.38 | 1.04 | 2.05 | 4.45E−02 |
| hsa-miR-320 | 5.88 | 4.85 | 1.03 | 2.05 | 7.88E−04 |
| hsa-miR-520a-3p | 0.23 | 1.24 | −1.02 | 2.02 | 1.60E−02 |
| hsa-miR-505* | 0.00 | −1.01 | 1.01 | 2.02 | 4.39E−02 |
| hsa-miR-572 | 6.94 | 5.93 | 1.01 | 2.01 | 3.85E−02 |
| hsa-miR-760 | 2.62 | 3.63 | −1.01 | 2.01 | 3.50E−02 |
| hsa-miR-139-3p | 2.18 | 3.18 | −1.00 | 2.00 | 3.05E−03 |
| hsa-miR-181b | 3.16 | 2.19 | 0.98 | 1.97 | 3.28E−03 |
| hsa-miR-605 | 1.28 | 2.26 | −0.97 | 1.96 | 3.91E−04 |
| hsa-miR-493 | 0.00 | 0.97 | −0.97 | 1.96 | 4.91E−02 |
| hsa-miR-371-3p | 0.58 | 1.53 | −0.95 | 1.93 | 6.09E−04 |
| hsa-miR-211 | 0.99 | 1.94 | −0.95 | 1.93 | 3.22E−02 |
| hsa-miR-877* | 3.95 | 3.00 | 0.95 | 1.93 | 1.62E−04 |
| hsa-miR-367 | −0.35 | 0.60 | −0.95 | 1.93 | 2.40E−02 |
| hsa-miR-296-5p | 4.97 | 4.05 | 0.92 | 1.89 | 2.45E−03 |
| hsa-miR-155 | 5.02 | 4.10 | 0.92 | 1.89 | 3.48E−02 |
| hsa-miR-526b* | 0.31 | 1.22 | −0.91 | 1.88 | 8.32E−03 |
| hsa-miR-193a-3p | 2.39 | 1.49 | 0.90 | 1.87 | 3.96E−02 |
| hsa-miR-1234 | 5.01 | 4.11 | 0.90 | 1.86 | 5.63E−03 |
| hsa-miR-563 | 3.27 | 2.38 | 0.89 | 1.85 | 4.55E−03 |
| hsa-miR-1237 | 3.91 | 3.03 | 0.88 | 1.84 | 1.48E−03 |
| hsa-miR-130b* | 0.39 | 1.15 | −0.76 | 1.69 | 3.42E−02 |
| hsa-miR-483-3p | 2.49 | 3.23 | −0.74 | 1.67 | 5.20E−03 |
| hsa-miR-767-3p | 2.17 | 1.43 | 0.74 | 1.67 | 1.02E−02 |
| hsa-miR-625* | 2.76 | 2.02 | 0.74 | 1.66 | 2.59E−03 |
| hsa-miR-940 | 6.43 | 5.70 | 0.73 | 1.66 | 1.43E−02 |
| hsa-miR-532-3p | 0.72 | 0.02 | 0.71 | 1.63 | 2.03E−02 |
| hsa-miR-502-3p | 0.89 | 0.19 | 0.70 | 1.63 | 3.25E−02 |
| hsa-miR-191* | 4.21 | 3.53 | 0.68 | 1.60 | 3.59E−02 |
| hsa-miR-1225-3p | 4.32 | 3.65 | 0.68 | 1.60 | 2.56E−02 |
| hsa-miR-937 | 0.81 | 1.42 | −0.61 | 1.53 | 4.61E−02 |
| hsa-miR-365 | 3.11 | 2.51 | 0.60 | 1.52 | 5.88E−03 |
| hsa-miR-520d-3p | 1.01 | 1.59 | −0.59 | 1.50 | 3.70E−02 |

The miRNAs identified in Tables 5, 6, 10, and 11 are useful as prognostic markers to predict if patients with Stage II colorectal cancer are likely to have cancer recurrence within a five-year period. The inventors performed data analysis across platforms and identified the miRNAs listed below as being common across the different platforms used. These miRNAs are particularly useful as prognostic markers to predict a patient's likelihood of colorectal cancer recurrence. miRNAs particularly useful as prognostic markers for predicting colorectal cancer recurrence include, but are not limited to hsa-miR-15b, hsa-miR-20b, hsa-miR-93, hsa-let-7f, hsa-miR-20a, hsa-miR-19b, hsa-miR-103, hsa-let-7g, hsa-miR-107, hsa-miR-25, hsa-miR-16, hsa-miR-128, hsa-miR-28-5p, hsa-miR-26b, hsa-miR-29a, hsa-miR-221, hsa-miR-29b-1*, hsa-miR-185, hsa-miR-34a, hsa-miR-148a, hsa-miR-146a, hsa-miR-155, hsa-miR-146b, hsa-miR-15a, hsa-let-71, hsa-miR-191, hsa-miR-501-5p, hsa-miR-632, hsa-miR-500, hsa-let-7c*, hsa-miR-125b-2*, hsa-miR-892b, hsa-miR-139-3p, hsa-miR-596, hsa-miR-135b*, hsa-miR-302c*, or hsa-miR-675.

### REFERENCES

The following references, to the extent that they provide exemplary procedural or other details supplementary to those set forth herein, are specifically incorporated herein by reference.
U.S. Pat. No. 4,337,063
U.S. Pat. No. 4,404,289
U.S. Pat. No. 4,405,711
U.S. Pat. No. 4,659,774
U.S. Pat. No. 4,682,195
U.S. Pat. No. 4,683,202
U.S. Pat. No. 4,704,362
U.S. Pat. No. 4,816,571
U.S. Pat. No. 4,959,463
U.S. Pat. No. 5,141,813
U.S. Pat. No. 5,143,854
U.S. Pat. No. 5,202,231
U.S. Pat. No. 5,214,136
U.S. Pat. No. 5,221,619
U.S. Pat. No. 5,223,618
U.S. Pat. No. 5,242,974
U.S. Pat. No. 5,264,566
U.S. Pat. No. 5,268,486
U.S. Pat. No. 5,288,644
U.S. Pat. No. 5,324,633
U.S. Pat. No. 5,378,825
U.S. Pat. No. 5,384,261
U.S. Pat. No. 5,405,783
U.S. Pat. No. 5,411,876
U.S. Pat. No. 5,412,087
U.S. Pat. No. 5,413,924
U.S. Pat. No. 5,424,186
U.S. Pat. No. 5,428,148
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,786
U.S. Pat. No. 5,468,613
U.S. Pat. No. 5,470,710
U.S. Pat. No. 5,470,967
U.S. Pat. No. 5,472,672
U.S. Pat. No. 5,480,980
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,550,044
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,580,726
U.S. Pat. No. 5,580,732
U.S. Pat. No. 5,583,013
U.S. Pat. No. 5,985,619
U.S. Pat. No. 5,593,839
U.S. Pat. No. 5,599,672
U.S. Pat. No. 5,599,695
U.S. Pat. No. 5,602,240
U.S. Pat. No. 5,602,244
U.S. Pat. No. 5,610,287
U.S. Pat. No. 5,610,289
U.S. Pat. No. 5,614,617
U.S. Pat. No. 5,623,070
U.S. Pat. No. 5,624,711
U.S. Pat. No. 5,631,134
U.S. Pat. No. 5,637,683
U.S. Pat. No. 5,639,603
U.S. Pat. No. 5,645,897
U.S. Pat. No. 5,652,099
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,670,663
U.S. Pat. No. 5,672,697
U.S. Pat. No. 5,681,947
U.S. Pat. No. 5,695,940
U.S. Pat. No. 5,700,637
U.S. Pat. No. 5,700,922
U.S. Pat. No. 5,705,629
U.S. Pat. No. 5,708,154
U.S. Pat. No. 5,714,606
U.S. Pat. No. 5,728,525
U.S. Pat. No. 5,744,305
U.S. Pat. No. 5,763,167
U.S. Pat. No. 5,777,092
U.S. Pat. No. 5,792,847
U.S. Pat. No. 5,800,992
U.S. Pat. No. 5,807,522
U.S. Pat. No. 5,830,645
U.S. Pat. No. 5,837,196
U.S. Pat. No. 5,847,219
U.S. Pat. No. 5,858,988
U.S. Pat. No. 5,859,221
U.S. Pat. No. 5,871,928
U.S. Pat. No. 5,872,232
U.S. Pat. No. 5,876,932
U.S. Pat. No. 5,886,165
U.S. Pat. No. 5,919,626
U.S. Pat. No. 6,004,755
U.S. Pat. No. 6,087,102
U.S. Pat. No. 6,251,666
U.S. Pat. No. 6,368,799
U.S. Pat. No. 6,383,749
U.S. Pat. No. 6,403,341
U.S. Pat. No. 6,617,112
U.S. Pat. No. 6,638,717
U.S. Pat. No. 6,720,138
U.S. Pat. No. 6,723,509
U.S. patent application Ser. No. 11/857,948
U.S. patent application Ser. No. 11/567,082
U.S. patent application Ser. No. 09/545,207
U.S. patent application Ser. No. 10/667,126
U.S. patent application Ser. No. 11/141,707
U.S. patent application Ser. No. 11/273,640
U.S. patent Prov. application Ser. No. 11/855,792
U.S. Patent Prov. Appn. Ser. No. 60/575,743
U.S. Patent Prov. Appn. Ser. No. 60/649,584
U.S. Patent Prov. Appn. Ser. No. 60/826,173
U.S. Patent Prov. Appn. Ser. No. 60/869,295
Akao et al., Biol Pharm Bull., 29(5):903-906, 2006.
Akao et al., DNA Cell Biol., 26:311-320, 2007.
Andersen et al., Cancer Res., 64(15):5245-5250, 2004.
Baddi and Benson, Oncologist, 10:325-331, 2005
Bandres et al., Molec. Cancer, 5:29, 2006.
Berezikov et al., Cell, 120(1):21-24, 2005.
Calin and Croce, Nat. Rev. Cancer, 6(11):857-866, 2006.
Carrington and Ambros, Science, 301(5631):336-338, 2003.
Chmielarz, et al., Med. Sci. Monit., 7:1263-1269, 2001
Crissman, et al., Pathol. Annu. 24(Pt 2):103-147, 1989.
Cummins et al., In: IRT: Nucleosides and nucleosides, La Jolla Calif., 72, 1996.
Cummins et al., Proc. Natl. Acad. Sci. USA, 103(10):3687-3692, 2006.
Davison et al., Meth Enzymol., 411:14-34, 2006.
Denli et al., Trends Biochem. Sci., 28:196, 2003.
Didenko, Biotechniques, 31(5):1106-16, 1118, 1120-1, 2001.
Emptage et al., Neuron, 29(1):197-208, 2001.
EP 266,032
EP 373 203
EP 785 280
EP 799 897
Esquela-Kerscher and Slack, Nat. Rev. Cancer, 6(4):259-269, 2006.
Froehler et al., Nucleic Acids Res., 14(13):5399-5407, 1986.
Greene, et al., (Eds.) AJCC Cancer Staging Manual, 6^{th} ed., Springer, New York, 2002.
Griffey et al., J Mass Spectrom, 32(3):305-13, 1997.
Griffiths-Jones et al., Nucleic Acids Res., 34:D140-D144, 2006.
Haller et al., J Clin Oncol., 23:8671-8678, 2005
Huber et al., Bioinformatics, 18(S1):S96-S104, 2002.
Itakura and Riggs, Science, 209:1401-1405, 1980.
Jemal et al., CA Cancer J. Clin., 57:43-66, 2007.
Klostermeier and Millar, Biopolymers, 61(3):159-79, 2001-2002.
Lanza et al., Molec Cancer, 6:54, 2007.
Lee and Ambros, Science, 294(5543):862-864, 2001.
MAQC Consortium, Nat Biotechnol., 24(9):1151-1161, 2006.
Meyerhardt and Mayer, New Engl. J. Med., 352:4764-87, 2005.
Meyerhardt and Mayer, Semin. Oncol., 30:349-360, 2003
Meyerhardt et al., Ann. Intern. Med., 139:649-657, 2003
Michael et al., Mol. Cancer. Res., 1:882-891, 2003.
Olsen et al., Dev. Biol., 216:671, 1999.
PCT Appln. WO 0138580
PCT Appln. WO 0168255
PCT Appln. WO 03020898
PCT Appln. WO 03022421
PCT Appln. WO 03023058
PCT Appln. WO 03029485
PCT Appln. WO 03040410
PCT Appln. WO 03053586
PCT Appln. WO 03066906
PCT Appln. WO 03067217
PCT Appln. WO 03076928
PCT Appln. WO 03087297
PCT Appln. WO 03091426
PCT Appln. WO 03093810
PCT Appln. WO 03100012
PCT Appln. WO 03100448A1
PCT Appln. WO 04020085
PCT Appln. WO 04027093
PCT Appln. WO 09923256
PCT Appln. WO 09936760
PCT Appln. WO 93/17126
PCT Appln. WO 95/11995
PCT Appln. WO 95/21265
PCT Appln. WO 95/21944
PCT Appln. WO 95/21944
PCT Appln. WO 95/35505
PCT Appln. WO 96/31622
PCT Appln. WO 97/10365
PCT Appln. WO 97/27317
PCT Appln. WO 9743450
PCT Appln. WO 99/35505
Pietras et al., Oncogene, 17(17):2235-2249, 1998.
Remington's Pharmaceutical Sciences” 15th Edition, pages 1035-1038 and 1570-1580, 1990.
Sambrook et al., In: DNA microaarays: a molecular cloning manual, Cold Spring Harbor Laboratory Press, Cold Spring Harbor, N.Y., 2003.
Sambrook et al., In: Molecular cloning: a laboratory manual, 2^{nd} Ed., Cold Spring Harbor Laboratory Press, Cold Spring Harbor, N.Y., 1989.
Sambrook et al., In: Molecular cloning: a laboratory manual, 3^{rd} Ed., Cold Spring Harbor Laboratory Press, Cold Spring Harbor, N.Y., 2001.
Sargent et al., J Clin Oncol. 23:8664-8670, 2005
Schetter et al., JAMA, 299(4):425-436, 2008.
Seggerson et al., Dev. Biol., 243:215, 2002.
Sobin and Wittekind, (Eds.) TNM Classification of Malignant Tumours, 6^{th} ed., John Wiley & Sons, New Jersey, 2002.
U.K. Patent 8 803 000
Xi et al., Biomark Insights, 2:113-121, 2006a.
Xi et al., Clin Cancer Res., 12:2014-2024, 2006b.
Xie, et al., Nature, 434(7031):338-345, 2005.

What is claimed is:

1. A method for evaluating a patient comprising the steps of:
(a) determining expression levels of one or more miRNA from Table 3, 4, 5, 6, 7, 10 and/or 11 in a biological sample comprising a portion of a suspect lesion taken from the patient using one or more oligonucleotides that specifically interact with the miRNA to detect the miRNA, and
(b) determining a diagnosis or prognosis for colorectal cancer based on the miRNA expression levels.

2. The method of claim 1, wherein one or more miRNA is selected from a group consisting of hsa-miR-15b, hsa-miR-20b, hsa-miR-93, hsa-let-7f, hsa-miR-20a, hsa-miR-19b, hsa-miR-103, hsa-let-7g, hsa-miR-107, hsa-miR-25, hsa-miR-16, hsa-miR-128, hsa-miR-28-5p, hsa-miR-26b, hsa-miR-29a, hsa-miR-221, hsa-miR-29b-1*, hsa-miR-185, hsa-miR-34a, hsa-miR-148a miR-146a miR-155 miR-146b miR-15a let-71 miR-191, hsa-miR-501-5p, hsa-miR-632, hsa-miR-500, hsa-let-7c*, hsa-miR-125b-2*, hsa-miR-892b, hsa-miR-139-3p, hsa-miR-596, hsa-miR-135b*, hsa-miR-302c*, and hsa-miR-675.

3. The method of claim 1, wherein the patient is suspected of having colorectal cancer.

4. The method of claim 1, wherein the patient is at risk of colorectal cancer recurrence.

5. The method of claim 1, wherein the cancer is colorectal cancer.

6. The method of claim 1, wherein determining a diagnosis is screening for a pathological condition, staging a pathological condition, or assessing response of a pathological condition to therapy.

7. The method of claim 6, wherein determining a diagnosis is determining if the patient has colorectal cancer.

8. The method of claim 1, further comprising normalizing the expression levels of miRNA.

9. The method of claim 8, wherein normalizing is adjusting expression levels of miRNA relative to expression levels of one or more nucleic acid in the sample.

10. The method of claim 1, further comprising comparing miRNA expression levels in the sample to miRNA expression levels in a normal tissue sample or reference.

11. The method of claim 10, wherein the sample from the patient and the normal tissue sample are colorectal samples.

12. The method of claim 10, wherein the normal tissue sample is not from the patient being evaluated.

13. The method of claim 11, wherein the normal tissue sample is taken from the patient being evaluated.

14. The method of claim 11, wherein the normal tissue sample is normal adjacent tissue.

15. The method of claim 1, wherein determining a prognosis involves estimating the likelihood of recurrence of colorectal cancer.

16. The method of claim 1, wherein expression of the miRNA is determined by an amplification assay or a hybridization assay.

17. The method of claim 16, wherein amplification assay is a quantitative amplification assay.

18. The method of claim 17, wherein the quantitative amplification assay is quantitative RT-PCR.

19. The method of claim 16, wherein the hybridization assay is an array hybridization assay or a solution hybridization assay.

20. The method of claim 1, further comprising providing a report of the diagnosis or prognosis.

21. The method of claim 1, further comprising obtaining a sample from the patient.

22. The method of claim 21, wherein expression levels of miRNA are determined without extracting RNA from the sample.

23. The method of claim 21, wherein expression levels of miRNA are determined after extracting RNA from the sample.

24. The method of claim 21 or 22, further comprising labeling miRNA to be detected.

25. The method of claim 1, wherein the sample is a tissue sample.

26. The method of claim 25, wherein the sample is fresh, frozen, fixed, or embedded.

27. The method of claim 26, wherein the sample is a formalin fixed, paraffin-embedded (FFPE) tissue.

28. A method for assessing the likelihood of colorectal cancer recurrence in a patient comprising the steps of:
(a) determining the expression levels of one or more miRNA from Table 5, 6, 7, 10, and/or 11 in a biological sample comprising colorectal cancer cells taken from the patient, and
(b) determining a prognosis for colorectal cancer recurrence based on the miRNA expression levels.

29. The method of claim 28, wherein the one or more miRNA is selected from a group consisting of hsa-miR-15b, hsa-miR-20b, hsa-miR-93, hsa-let-7f, hsa-miR-20a, hsa-miR-19b, hsa-miR-103, hsa-let-7g, hsa-miR-107, hsa-miR-25, hsa-miR-16, hsa-miR-128, hsa-miR-28-5p, hsa-miR-26b, hsa-miR-29a, hsa-miR-221, hsa-miR-29b-1*, hsa-miR-185, hsa-miR-34a, hsa-miR-148a miR-146a miR-155 miR-146b miR-15a let-71 miR-191, hsa-miR-501-5p, hsa-miR-632, hsa-miR-500, hsa-let-7c*, hsa-miR-125b-2*, hsa-miR-892b, hsa-miR-139-3p, hsa-miR-596, hsa-miR-135b*, hsa-miR-302c*, and hsa-miR-675.

30. The method of claim 28, wherein the patient is at risk of colorectal cancer recurrence.

31. The method of claim 28, further comprising normalizing the expression levels of the miRNA relative to at least a second nucleic acid in the sample.

32. The method of claim 28, wherein cancer is colon cancer.

33. The method of claim 28, wherein recurrence is a second instance of cancer within colon or rectal tissues of the patient, or in tissues adjacent to the colon or rectum of the patient after a first instance of colorectal cancer has been treated.

34. The method of claim 28, wherein recurrence is a second instance of cancer within non-colon or non-rectal tissues distant from a first instance of cololrectal cancer.

35. The method of claim 33 or 34, wherein the first instance of colorectal cancer is Stage I, Stage II, Stage III, or Stage IV cancer.

36. The method of claim 28, further comprising comparing miRNA expression levels to a reference.

37. The method of claim 36, wherein the reference is a sample from a patient comprising cancer cells, wherein the patient has been diagnosed with colorectal cancer and has not had a recurrence of colorectal cancer.

38. The method of claim 36, wherein the reference is a comparative dataset.

39. The method of claim 28, wherein the sample is fresh, frozen, fixed, or embedded.

40. The method of claim 39, wherein the sample is a formalin fixed, paraffin-embedded (FFPE) tissue.

41. The method of claim 39, wherein sample is a frozen tissue.

42. The method of claim 28, further comprising comparing miRNA expression levels in the biological sample to miRNA expression levels in a normal colorectal tissue or a cancerous colorectal tissue.

43. The method of claim 42, wherein the sample from the patient and the normal sample are colorectal samples.

44. The method of claim 42, wherein the normal tissue is from a patient that has had a recurrence of colorectal cancer.

45. The method of claim 42, wherein the normal sample is normal adjacent tissue.

46. The method of claim 28, further comprising obtaining a sample from the patient.

47. The method of claim 46, further comprising extracting RNA from the sample.

48. The method of claim 46 or 47, further comprising labeling miRNA from the sample.

49. The method of claim 28, wherein expression of the miRNA is determined by an amplification assay or a hybridization assay.

50. The method of claim 49, wherein amplification assay is a quantitative amplification assay.

51. The method of claim 50, wherein the quantitative amplification assay is quantitative RT-PCR.

52. The method of claim 49, wherein the hybridization assay is an array hybridization assay or a solution hybridization assay.

53. The method of claim 28, further comprising providing a report of the diagnosis and/or prognosis.

54. A kit for analysis of a sample by assessing miRNA profile for a sample comprising, in suitable container means, two or more miRNA hybridization or amplification reagents comprising one or more probe or amplification primer for one or more miRNA selected form Table 3, 4, 5, 6, 7, 10 and/or 11.

55. The kit of claim 54, wherein the one or more miRNA is selected from a group consisting of hsa-miR-15b, hsa-miR-20b, hsa-miR-93, hsa-let-7f, hsa-miR-20a, hsa-miR-19b, hsa-miR-103, hsa-let-7g, hsa-miR-107, hsa-miR-25, hsa-miR-16, hsa-miR-128, hsa-miR-28-5p, hsa-miR-26b, hsa-miR-29a, hsa-miR-221, hsa-miR-29b-1*, hsa-miR-185, hsa-miR-34a, hsa-miR-148a miR-146a miR-155 miR-146b miR-15a let-71 miR-191, hsa-miR-501-5p, hsa-miR-632, hsa-miR-500, hsa-let-7c*, hsa-miR-125b-2*, hsa-miR-892b, hsa-miR-139-3p, hsa-miR-596, hsa-miR-135b*, hsa-miR-302c*, and hsa-miR-675.

56. The kit of claim 54, further comprising reagents for detecting an miRNA in the sample.

57. The kit of claim 54, wherein miRNA hybridization reagent comprises hybridization probes.

58. The kit of claim 54, wherein miRNA amplification reagent comprises one or more of amplification primers or a probe for the detection of an miRNA sequence.


SEQUENCE LISTING (334 sequences)

SEQ ID NO: 1 - RNA, 21 nt, Homo sapiens
ugcaauguuaaaagggcauug

SEQ ID NO: 2 - RNA, 95 nt, Homo sapiens
gccucugcugcuggccagagcucuuuucacauugugcuacugucugcaccugucacuagcagugcaauguuaaaagggcauuggccguguagugc

SEQ ID NO: 3 - RNA, 21 nt, Homo sapiens
ugggcggacacgacauucccg

SEQ ID NO: 4 - RNA, 87 nt, Homo sapiens
gaaagagcacugggcggacacgacauucccgauggcuucucgggugcccacucaacgggagugaucgugucauuccaaagcgcuuuc

SEQ ID NO: 5 - RNA, 21 nt, Homo sapiens
uccaacaauauccuggugcug

SEQ ID NO: 6 - RNA, 96 nt, Homo sapiens
gccgcacggcuguccucuccaacaauauccuggugcugagugaugacucaggcgacuccagcaucagugauuuuguugaagagggcagcugccagc

SEQ ID NO: 7 - RNA, 21 nt, Homo sapiens
uugcacuugucccggccugug

SEQ ID NO: 8 - RNA, 92 nt, Homo sapiens
augcccauucaucccuggguggggauuuguugcauuacuuguguucuauauaaaguauugcacuugucccggccuguggaagaaaggaggau

SEQ ID NO: 9 - RNA, 22 nt, Homo sapiens
ugagguaguagauuguauaguu

SEQ ID NO: 10 - RNA, 83 nt, Homo sapiens
ugugggaugagguaguagauuguauaguuuuagggucauaccccaucuuggagauaacuauacagucuacugucuuucccacg

SEQ ID NO: 11 - RNA, 21 nt, Homo sapiens
ugagguaguaguuuguacagu

SEQ ID NO: 12 - RNA, 84 nt, Homo sapiens
aggcugagguaguaguuuguacaguuugagggucuaugauaccacccgguacaggagauaacuguacaggccacugccuugcca

SEQ ID NO: 13 - RNA, 21 nt, Homo sapiens
ugagguaguaguuugugcugu

SEQ ID NO: 14 - RNA, 84 nt, Homo sapiens
cuggcugagguaguaguuugugcuguuggucggguugugacauugcccgcuguggagauaacugcgcaagcuacugccuugcua

SEQ ID NO: 15 - RNA, 21 nt, Homo sapiens
uggaauguaaagaaguaugua

SEQ ID NO: 16 - RNA, 71 nt, Homo sapiens
ugggaaacauacuucuuuauaugcccauauggaccugcuaagcuauggaauguaaagaaguauguaucuca

SEQ ID NO: 17 - RNA, 23 nt, Homo sapiens
agcagcauuguacagggcuauga

SEQ ID NO: 18 - RNA, 78 nt, Homo sapiens
uacugcccucggcuucuuuacagugcugccuuguugcauauggaucaagcagcauuguacagggcuaugaaggcauug

SEQ ID NO: 19 - RNA, 24 nt, Homo sapiens
aaaagugcuuacagugcagguagc

SEQ ID NO: 20 - RNA, 81 nt, Homo sapiens
ccuuggccauguaaaagugcuuacagugcagguagcuuuuugagaucuacugcaauguaagcacuucuuacauuaccaugg

SEQ ID NO: 21 - RNA, 21 nt, Homo sapiens
uaaagugcugacagugcagau

SEQ ID NO: 22 - RNA, 82 nt, Homo sapiens
ccugccggggcuaaagugcugacagugcagauagugguccucuccgugcuaccgcacuguggguacuugcugcuccagcagg

SEQ ID NO: 23 - RNA, 23 nt, Homo sapiens
agcagcauuguacagggcuauca

SEQ ID NO: 24 - RNA, 81 nt, Homo sapiens
cucucugcuuucagcuucuuuacaguguugccuuguggcauggaguucaagcagcauuguacagggcuaucaaagcacaga

SEQ ID NO: 25 - RNA, 22 nt, Homo sapiens
uacccuguagaaccgaauuugu

SEQ ID NO: 26 - RNA, 110 nt, Homo sapiens
ccagagguuguaacguugucuauauauacccuguagaaccgaauuugugugguauccguauagucacagauucgauucuaggggaauauauggucgaugcaaaaacuuca

SEQ ID NO: 27 - RNA, 22 nt, Homo sapiens
ucacagugaaccggucucuuuu

SEQ ID NO: 28 - RNA, 82 nt, Homo sapiens
ugagcuguuggauucggggccguagcacugucugagagguuuacauuucucacagugaaccggucucuuuuucagcugcuuc

SEQ ID NO: 29 - RNA, 22 nt, Homo sapiens
cagugcaauguuaaaagggcau

SEQ ID NO: 30 - RNA, 89 nt, Homo sapiens
ugcugcuggccagagcucuuuucacauugugcuacugucugcaccugucacuagcagugcaauguuaaaagggcauuggccguguagug

SEQ ID NO: 31 - RNA, 22 nt, Homo sapiens
cagugcaaugaugaaagggcau

SEQ ID NO: 32 - RNA, 82 nt, Homo sapiens
ggccugcccgacacucuuucccuguugcacuacuauaggccgcugggaagcagugcaaugaugaaagggcaucggucagguc

SEQ ID NO: 33 - RNA, 22 nt, Homo sapiens
uugguccccuucaaccagcugu

SEQ ID NO: 34 - RNA, 102 nt, Homo sapiens
gggagccaaaugcuuugcuagagcugguaaaauggaaccaaaucgacuguccaauggauuugguccccuucaaccagcuguagcugugcauugauggcgccg

SEQ ID NO: 35 - RNA, 21 nt, Homo sapiens
uugguccccuucaaccagcua

SEQ ID NO: 36 - RNA, 119 nt, Homo sapiens
ccucagaagaaagaugcccccugcucuggcuggucaaacggaaccaaguccgucuuccugagagguuugguccccuucaaccagcuacagcagggcuggcaaugcccaguccuuggaga

SEQ ID NO: 37 - RNA, 22 nt, Homo sapiens
uaacacugucugguaaagaugg

SEQ ID NO: 38 - RNA, 95 nt, Homo sapiens
cggccggcccuggguccaucuuccaguacaguguuggauggucuaauugugaagcuccuaacacugucugguaaagauggcucccggguggguuc

SEQ ID NO: 39 - RNA, 22 nt, Homo sapiens
ugagaugaagcacuguagcuca

SEQ ID NO: 40 - RNA, 106 nt, Homo sapiens
gcgcagcgcccugucucccagccugaggugcagugcugcaucucuggucaguugggagucugagaugaagcacuguagcucaggaagagagaaguuguucugcagc

SEQ ID NO: 41 - RNA, 24 nt, Homo sapiens
guccaguuuucccaggaaucccuu

SEQ ID NO: 42 - RNA, 88 nt, Homo sapiens
caccuuguccucacgguccaguuuucccaggaaucccuuagaugcuaagauggggauuccuggaaauacuguucuugaggucaugguu

SEQ ID NO: 43 - RNA, 22 nt, Homo sapiens
ugagaacugaauuccauggguu

SEQ ID NO: 44 - RNA, 99 nt, Homo sapiens
ccgauguguauccucagcuuugagaacugaauuccauggguugugucagugucagaccucugaaauucaguucuucagcugggauaucucugucaucgu

SEQ ID NO: 45 - RNA, 22 nt, Homo sapiens
ugagaacugaauuccauaggcu

SEQ ID NO: 46 - RNA, 73 nt, Homo sapiens
ccuggcacugagaacugaauuccauaggcugugagcucuagcaaugcccuguggacucaguucuggugcccgg

SEQ ID NO: 47 - RNA, 22 nt, Homo sapiens
ucagugcacuacagaacuuugu

SEQ ID NO: 48 - RNA, 68 nt, Homo sapiens
gaggcaaaguucugagacacuccgacucugaguaugauagaagucagugcacuacagaacuuugucuc

SEQ ID NO: 49 - RNA, 22 nt, Homo sapiens
ucucccaacccuuguaccagug

SEQ ID NO: 50 - RNA, 84 nt, Homo sapiens
cuccccauggcccugucucccaacccuuguaccagugcugggcucagacccugguacaggccugggggacagggaccuggggac

SEQ ID NO: 51 - RNA, 22 nt, Homo sapiens
ucagugcaugacagaacuuggg

SEQ ID NO: 52 - RNA, 87 nt, Homo sapiens
ugucccccccggcccagguucugugauacacuccgacucgggcucuggagcagucagugcaugacagaacuugggcccggaaggacc

SEQ ID NO: 53 - RNA, 22 nt, Homo sapiens
uuaaugcuaaucgugauagggg

SEQ ID NO: 54 - RNA, 65 nt, Homo sapiens
cuguuaaugcuaaucgugauagggguuuuugccuccaacugacuccuacauauuagcauuaacag

SEQ ID NO: 55 - RNA, 22 nt, Homo sapiens
uagcagcacauaaugguuugug

SEQ ID NO: 56 - RNA, 83 nt, Homo sapiens
ccuuggaguaaaguagcagcacauaaugguuuguggauuuugaaaaggugcaggccauauugugcugccucaaaaauacaagg

SEQ ID NO: 57 - RNA, 22 nt, Homo sapiens
uagcagcacaucaugguuuaca

SEQ ID NO: 58 - RNA, 98 nt, Homo sapiens
uugaggccuuaaaguacuguagcagcacaucaugguuuacaugcuacagucaagaugcgaaucauuauuugcugcucuagaaauuuaaggaaauucau

SEQ ID NO: 59 - RNA, 22 nt, Homo sapiens
uagcagcacguaaauauuggcg

SEQ ID NO: 60 - RNA, 81 nt, Homo sapiens
guuccacucuagcagcacguaaauauuggcguagugaaauauauauuaaacaccaauauuacugugcugcuuuagugugac

SEQ ID NO: 61 - RNA, 20 nt, Homo sapiens
acugcagugaaggcacuugu

SEQ ID NO: 62 - RNA, 84 nt, Homo sapiens
gucagaauaaugucaaagugcuuacagugcagguagugauaugugcaucuacugcagugaaggcacuuguagcauuauggugac

SEQ ID NO: 63 - RNA, 24 nt, Homo sapiens
caaagugcuuacagugcagguagu

SEQ ID NO: 64 - RNA, 84 nt, Homo sapiens; identical to SEQ ID NO: 62

SEQ ID NO: 65 - RNA, 24 nt, Homo sapiens
aacauucauuguugucgguggguu

SEQ ID NO: 66 - RNA, 137 nt, Homo sapiens
guccccuccccuaggccacagccgaggucacaaucaacauucauuguugucgguggguugugaggacugaggccagacccaccgggggaugaaugucacuguggcugggccagacacggcuuaaggggaauggggac

SEQ ID NO: 67 - RNA, 22 nt, Homo sapiens
uuuggcaaugguagaacucaca

SEQ ID NO: 68 - RNA, 110 nt, Homo sapiens
gagcugcuugccuccccccguuuuuggcaaugguagaacucacacuggugagguaacaggauccggugguucuagacuugccaacuauggggcgaggacucagccggcac

SEQ ID NO: 69 - RNA, 23 nt, Homo sapiens
uauggcacugguagaauucacug

SEQ ID NO: 70 - RNA, 110 nt, Homo sapiens
ccgcagagugugacuccuguucuguguauggcacugguagaauucacugugaacagucucagucagugaauuaccgaagggccauaaacagagcagagacagauccacga

SEQ ID NO: 71 - RNA, 18 nt, Homo sapiens
uggagagaaaggcaguuc

SEQ ID NO: 72 - RNA, 82 nt, Homo sapiens
agggggcgagggauuggagagaaaggcaguuccugaugguccccuccccaggggcuggcuuuccucugguccuucccuccca

SEQ ID NO: 73 - RNA, 22 nt, Homo sapiens
caacggaaucccaaaagcagcu

SEQ ID NO: 74 - RNA, 92 nt, Homo sapiens
cggcuggacagcgggcaacggaaucccaaaagcagcuguugucuccagagcauuccagcugcgcuuggauuucguccccugcucuccugccu

SEQ ID NO: 75 - RNA, 21 nt, Homo sapiens
cugaccuaugaauugacagcc

SEQ ID NO: 76 - RNA, 110 nt, Homo sapiens
gccgagaccgagugcacagggcucugaccuaugaauugacagccagugcucucgucuccccucuggcugccaauuccauaggucacagguauguucgccucaaugccagc

SEQ ID NO: 77 - RNA, 22 nt, Homo sapiens
uguaacagcaacuccaugugga

SEQ ID NO: 78 - RNA, 85 nt, Homo sapiens
ugguucccgcccccuguaacagcaacuccauguggaagugcccacugguuccaguggggcugcuguuaucuggggcgagggccag

SEQ ID NO: 79 - RNA, 21 nt, Homo sapiens
uagcagcacagaaauauuggc

SEQ ID NO: 80 - RNA, 87 nt, Homo sapiens
agcuucccuggcucuagcagcacagaaauauuggcacagggaagcgagucugccaauauuggcugugcugcuccaggcaggguggug

SEQ ID NO: 81 - RNA, 21 nt, Homo sapiens
uagguaguuucauguuguugg

SEQ ID NO: 82 - RNA, 110 nt, Homo sapiens
ugcucgcucagcugaucuguggcuuagguaguuucauguuguugggauugaguuuugaacucggcaacaagaaacugccugaguuacaucagucgguuuucgucgagggc

SEQ ID NO: 83 - RNA, 21 nt, Homo sapiens
uagguaguuuccuguuguugg

SEQ ID NO: 84 - RNA, 84 nt, Homo sapiens
acuggucggugauuuagguaguuuccuguuguugggauccaccuuucucucgacagcacgacacugccuucauuacuucaguug

SEQ ID NO: 85 - RNA, 23 nt, Homo sapiens
ugugcaaauccaugcaaaacuga

SEQ ID NO: 86 - RNA, 96 nt, Homo sapiens
acauugcuacuuacaauuaguuuugcagguuugcauuucagcguauauauguauauguggcugugcaaauccaugcaaaacugauugugauaaugu

SEQ ID NO: 87 - RNA, 22 nt, Homo sapiens
gugaaauguuuaggaccacuag

SEQ ID NO: 88 - RNA, 110 nt, Homo sapiens
guguuggggacucgcgcgcuggguccagugguucuuaacaguucaacaguucuguagcgcaauugugaaauguuuaggaccacuagacccggcgggcgcggcgacagcga

SEQ ID NO: 89 - RNA, 22 nt, Homo sapiens
uccuucauuccaccggagucug

SEQ ID NO: 90 - RNA, 110 nt, Homo sapiens
aaagauccucagacaauccaugugcuucucuuguccuucauuccaccggagucugucucauacccaaccagauuucaguggagugaaguucaggaggcauggagcugaca

SEQ ID NO: 91 - RNA, 23 nt, Homo sapiens
uaaagugcuuauagugcagguag

SEQ ID NO: 92 - RNA, 71 nt, Homo sapiens
guagcacuaaagugcuuauagugcagguaguguuuaguuaucuacugcauuaugagcacuuaaaguacugc

SEQ ID NO: 93 - RNA, 23 nt, Homo sapiens
caaagugcucauagugcagguag

SEQ ID NO: 94 - RNA, 69 nt, Homo sapiens
aguaccaaagugcucauagugcagguaguuuuggcaugacucuacuguaguaugggcacuuccaguacu

SEQ ID NO: 95 - RNA, 22 nt, Homo sapiens
uagcuuaucagacugauguuga

SEQ ID NO: 96 - RNA, 72 nt, Homo sapiens
ugucggguagcuuaucagacugauguugacuguugaaucucauggcaacaccagucgaugggcugucugaca

SEQ ID NO: 97 - RNA, 21 nt, Homo sapiens
augaccuaugaauugacagac

SEQ ID NO: 98 - RNA, 110 nt, Homo sapiens
aucauucagaaaugguauacaggaaaaugaccuaugaauugacagacaauauagcugaguuugucugucauuucuuuaggccaauauucuguaugacugugcuacuucaa

SEQ ID NO: 99 - RNA, 21 nt, Homo sapiens
uugugcuugaucuaaccaugu

SEQ ID NO: 100 - RNA, 110 nt, Homo sapiens
gaccagucgcugcggggcuuuccuuugugcuugaucuaaccaugugguggaacgauggaaacggaacaugguucugucaagcaccgcggaaagcaccgugcucuccugca

SEQ ID NO: 101 - RNA, 23 nt, Homo sapiens
agcuacauugucugcuggguuuc

SEQ ID NO: 102 - RNA, 110 nt, Homo sapiens
ugaacauccaggucuggggcaugaaccuggcauacaauguagauuucuguguucguuaggcaacagcuacauugucugcuggguuucaggcuaccuggaaacauguucuc

SEQ ID NO: 103 - RNA, 24 nt, Homo sapiens
agcuacaucuggcuacugggucuc

SEQ ID NO: 104 - RNA, 110 nt, Homo sapiens
gcugcuggaagguguagguacccucaauggcucaguagccaguguagauccugucuuucguaaucagcagcuacaucuggcuacugggucucugauggcaucuucuagcu

SEQ ID NO: 105 - RNA, 21 nt, Homo sapiens
ugucaguuugucaaauacccc

SEQ ID NO: 106 - RNA, 110 nt, Homo sapiens
ccuggccuccugcagugccacgcuccguguauuugacaagcugaguuggacacuccaugugguagagugucaguuugucaaauaccccaagugcggcacaugcuuaccag

SEQ ID NO: 107 - RNA, 23 nt, Homo sapiens
caagucacuagugguuccguuua

SEQ ID NO: 108 - RNA, 81 nt, Homo sapiens
gggcuuucaagucacuagugguuccguuuaguagaugauugugcauuguuucaaaauggugcccuagugacuacaaagccc

SEQ ID NO: 109 - RNA, 21 nt, Homo sapiens
aucacauugccagggauuucc

SEQ ID NO: 110 - RNA, 73 nt, Homo sapiens
ggccggcugggguuccuggggaugggauuugcuuccugucacaaaucacauugccagggauuuccaaccgacc

SEQ ID NO: 111 - RNA, 22 nt, Homo sapiens
cauugcacuugucucggucuga

SEQ ID NO: 112 - RNA, 84 nt, Homo sapiens
ggccaguguugagaggcggagacuugggcaauugcuggacgcugcccugggcauugcacuugucucggucugacagugccggcc

SEQ ID NO: 113 - RNA, 22 nt, Homo sapiens
uauaauacaaccugcuaagugu

SEQ ID NO: 114 - RNA, 105 nt, Homo sapiens
agaaauccuacucggauggauauaauacaaccugcuaaguguccuagcacuuagcagguuguauuaucauuguccgugucuauggcucucgucuaccagacuuua

SEQ ID NO: 115 - RNA, 22 nt, Homo sapiens
uucaaguaauucaggauagguu

SEQ ID NO: 116 - RNA, 77 nt, Homo sapiens
ccgggacccaguucaaguaauucaggauagguugugugcuguccagccuguucuccauuacuuggcucggggaccgg

SEQ ID NO: 117 - RNA, 21 nt, Homo sapiens
uucacaguggcuaaguuccgc

SEQ ID NO: 118 - RNA, 78 nt, Homo sapiens
cugaggagcagggcuuagcugcuugugagcaggguccacaccaagucguguucacaguggcuaaguuccgccccccag

SEQ ID NO: 119 - RNA, 21 nt, Homo sapiens
uucacaguggcuaaguucugc

SEQ ID NO: 120 - RNA, 97 nt, Homo sapiens
accucucuaacaaggugcagagcuuagcugauuggugaacagugauugguuuccgcuuuguucacaguggcuaaguucugcaccugaagagaaggug

SEQ ID NO: 121 - RNA, 22 nt, Homo sapiens
aaggagcucacagucuauugag

SEQ ID NO: 122 - RNA, 86 nt, Homo sapiens
gguccuugcccucaaggagcucacagucuauugaguuaccuuucugacuuucccacuagauugugagcuccuggagggcaggcacu

SEQ ID NO: 123 - RNA, 21 nt, Homo sapiens
uagcaccaucugaaaucgguu

SEQ ID NO: 124 - RNA, 64 nt, Homo sapiens
augacugauuucuuuugguguucagagucaauauaauuuucuagcaccaucugaaaucgguuau

SEQ ID NO: 125 - RNA, 23 nt, Homo sapiens
uagcaccauuugaaaucaguguu

SEQ ID NO: 126 - RNA, 81 nt, Homo sapiens
cuucuggaagcugguuucacaugguggcuuagauuuuuccaucuuuguaucuagcaccauuugaaaucaguguuuuaggag

SEQ ID NO: 127 - RNA, 22 nt, Homo sapiens
auugcacucgucccggccuccg

SEQ ID NO: 128 - RNA, 111 nt, Homo sapiens
gcgggaucccgggccccgggcgggcgggagggacgggacgcggugcaguguuguuuuuucccccgccaauauugcacucgucccggccuccggcccccccggccccccggc

SEQ ID NO: 129 - RNA, 22 nt, Homo sapiens
cuuucagucggauguuugcagc

SEQ ID NO: 130 - RNA, 71 nt, Homo sapiens
gcgacuguaaacauccucgacuggaagcugugaagccacagaugggcuuucagucggauguuugcagcugc

SEQ ID NO: 131 - RNA, 22 nt, Homo sapiens
uguaaacauccucgacuggaag

SEQ ID NO: 132 - RNA, 71 nt, Homo sapiens; identical to SEQ ID NO: 130

SEQ ID NO: 133 - RNA, 22 nt, Homo sapiens
uguaaacauccuacacucagcu

SEQ ID NO: 134 - RNA, 88 nt, Homo sapiens
accaaguuucaguucauguaaacauccuacacucagcuguaauacauggauuggcugggagguggauguuuacuucagcugacuugga

SEQ ID NO: 135 - RNA, 23 nt, Homo sapiens
uguaaacauccuacacucucagc

SEQ ID NO: 136 - RNA, 89 nt, Homo sapiens
accaugcuguaguguguguaaacauccuacacucucagcugugagcucaagguggcugggagaggguuguuuacuccuucugccaugga

SEQ ID NO: 137 - RNA, 20 nt, Homo sapiens
uguaaacauccuugacugga

SEQ ID NO: 138 - RNA, 92 nt, Homo sapiens
gggcagucuuugcuacuguaaacauccuugacuggaagcuguaagguguucagaggagcuuucagucggauguuuacagcggcaggcugcca

SEQ ID NO: 139 - RNA, 21 nt, Homo sapiens
ggcaagaugcuggcauagcug

SEQ ID NO: 140 - RNA, 71 nt, Homo sapiens
ggagaggaggcaagaugcuggcauagcuguugaacugggaaccugcuaugccaacauauugccaucuuucc

SEQ ID NO: 141 - RNA, 23 nt, Homo sapiens
ucaagagcaauaacgaaaaaugu

SEQ ID NO: 142 - RNA, 94 nt, Homo sapiens
uguuuugagcgggggucaagagcaauaacgaaaaauguuugucauaaaccguuuuucauuauugcuccugaccuccucucauuugcuauauuca

SEQ ID NO: 143 - RNA, 24 nt, Homo sapiens
ucucacacagaaaucgcacccguc

SEQ ID NO: 144 - RNA, 99 nt, Homo sapiens
gaaacugggcucaaggugaggggugcuaucugugauugagggacaugguuaauggaauugucucacacagaaaucgcacccgucaccuuggccuacuua

SEQ ID NO: 145 - RNA, 23 nt, Homo sapiens
uggcagugucuuagcugguuguu

SEQ ID NO: 146 - RNA, 110 nt, Homo sapiens
ggccagcugugaguguuucuuuggcagugucuuagcugguuguugugagcaauaguaaggaagcaaucagcaaguauacugcccuagaagugcugcacguuguggggccc

SEQ ID NO: 147 - RNA, 22 nt, Homo sapiens
uuaucagaaucuccagggguac

SEQ ID NO: 148 - RNA, 72 nt, Homo sapiens
ggagcuuaucagaaucuccagggguacuuuauaauuucaaaaagucccccaggugugauucugauuugcuuc

SEQ ID NO: 149 - RNA, 22 nt, Homo sapiens
uuuguucguucggcucgcguga

SEQ ID NO: 150 - RNA, 64 nt, Homo sapiens
ccccgcgacgagccccucgcacaaaccggaccugagcguuuuguucguucggcucgcgugaggc

SEQ ID NO: 151 - RNA, 22 nt, Homo sapiens
cuggacuuagggucagaaggcc

SEQ ID NO: 152 - RNA, 90 nt, Homo sapiens
gagagaagcacuggacuuagggucagaaggccugagucucucugcugcagaugggcucucugucccugagccaagcuuuguccucccugg

SEQ ID NO: 153 - RNA, 22 nt, Homo sapiens
cuggacuuggagucagaaggcc

SEQ ID NO: 154 - RNA, 66 nt, Homo sapiens
agggcuccugacuccagguccuguguguuaccuagaaauagcacuggacuuggagucagaaggccu

SEQ ID NO: 155 - RNA, 22 nt, Homo sapiens
agcagcacacugugguuuguac

SEQ ID NO: 156 - RNA, 105 nt, Homo sapiens
cccgguccugcucccgccccagcagcacacugugguuuguacggcacuguggccacguccaaaccacacugugguguuagagcgagggugggggaggcaccgccg

SEQ ID NO: 157 - RNA, 23 nt, Homo sapiens
aaaccguuaccauuacugaguuu

SEQ ID NO: 158 - RNA, 72 nt, Homo sapiens
cuugggaauggcaaggaaaccguuaccauuacugaguuuaguaaugguaaugguucucuugcuauacccaga

SEQ ID NO: 159 - RNA, 21 nt, Homo sapiens
cagcagcacacugugguuugu

SEQ ID NO: 160 - RNA, 112 nt, Homo sapiens
ccaccccgguccugcucccgccccagcagcacacugugguuuguacggcacuguggccacguccaaaccacacugugguguuagagcgagggugggggaggcaccgccgagg

SEQ ID NO: 161 - RNA, 25 nt, Homo sapiens
cccaucugggguggccugugacuuu

SEQ ID NO: 162 - RNA, 89 nt, Homo sapiens
cuaauggauaaggcauuggccuccuaagccagggauuguggguucgagucccaucugggguggccugugacuuuuguccuuuuuucccc

SEQ ID NO: 163 - RNA, 22 nt, Homo sapiens
agggggaaaguucuauaguccu

SEQ ID NO: 164 - RNA, 85 nt, Homo sapiens
aggguagagggaugagggggaaaguucuauaguccuguaauuagaucucaggacuauagaacuuucccccucaucccucugcccu

SEQ ID NO: 165 - RNA, 23 nt, Homo sapiens
aauggcgccacuaggguugugca

SEQ ID NO: 166 - RNA, 98 nt, Homo sapiens
acgaauggcuaugcacugcacaacccuaggagagggugccauucacauagacuauaauugaauggcgccacuaggguugugcagugcacaaccuacac

SEQ ID NO: 167 - RNA, 22 nt, Homo sapiens
uacccauugcauaucggaguug

SEQ ID NO: 168 - RNA, 97 nt, Homo sapiens
cugcuccuucucccauacccauugcauaucggaguugugaauucucaaaacaccuccugugugcauggauuacaggagggugagccuugucaucgug

SEQ ID NO: 169 - RNA, 22 nt, Homo sapiens
uggaagacuagugauuuuguug

SEQ ID NO: 170 - RNA, 110 nt, Homo sapiens
agauuagaguggcuguggucuagugcuguguggaagacuagugauuuuguuguucugauguacuacgacaacaagucacagccggccucauagcgcagacucccuucgac

SEQ ID NO: 171 - RNA, 22 nt, Homo sapiens
aaagugcuguucgugcagguag

SEQ ID NO: 172 - RNA, 80 nt, Homo sapiens
cugggggcuccaaagugcuguucgugcagguagugugauuacccaaccuacugcugagcuagcacuucccgagcccccgg

SEQ ID NO: 173 - RNA, 22 nt, Homo sapiens
uucaacggguauuuauugagca

SEQ ID NO: 174 - RNA, 81 nt, Homo sapiens
aacacagugggcacucaauaaaugucuguugaauugaaaugcguuacauucaacggguauuuauugagcacccacucugug

SEQ ID NO: 175 - RNA, 22 nt, Homo sapiens
ugagguaguaaguuguauuguu

SEQ ID NO: 176 - RNA, 119 nt, Homo sapiens
aggauucugcucaugccagggugagguaguaaguuguauuguugugggguagggauauuaggccccaauuagaagauaacuauacaacuuacuacuuucccugguguguggcauauuca

SEQ ID NO: 177 - RNA, 22 nt, Homo sapiens
ugagguaguagguuguauaguu

SEQ ID NO: 178 - RNA, 22 nt, Homo sapiens
ugagguaguagguugugugguu

SEQ ID NO: 179 - RNA, 22 nt, Homo sapiens
ugagguaguagguuguaugguu

SEQ ID NO: 180 - RNA, 22 nt, Homo sapiens
uagaguuacacccugggaguua

SEQ ID NO: 181 - RNA, 22 nt, Homo sapiens
agagguaguagguugcauaguu

SEQ ID NO: 182 - RNA, 22 nt, Homo sapiens
cuauacgaccugcugccuuucu

SEQ ID NO: 183 - RNA, 22 nt, Homo sapiens
ugagguaggagguuguauaguu

SEQ ID NO: 184 - RNA, 22 nt, Homo sapiens
ugagguaguagauuguauaguu

SEQ ID NO: 185 - RNA, 22 nt, Homo sapiens
aacccguagauccgaacuugug

SEQ ID NO: 186 - RNA, 23 nt, Homo sapiens
uacccuguagauccgaauuugug

SEQ ID NO: 187 - RNA, 21 nt, Homo sapiens
ccccaccuccucucuccucag

SEQ ID NO: 188 - RNA, 22 nt, Homo sapiens
ugagccccugugccgcccccag

SEQ ID NO: 189 - RNA, 26 nt, Homo sapiens
gugagggcaugcaggccuggaugggg

SEQ ID NO: 190 - RNA, 20 nt, Homo sapiens
ucacaccugccucgcccccc

SEQ ID NO: 191 - RNA, 22 nt, Homo sapiens
ucggccugaccacccaccccac

SEQ ID NO: 192 - RNA, 21 nt, Homo sapiens
uccuucugcuccgucccccag

SEQ ID NO: 193 - RNA, 20 nt, Homo sapiens
uaaggcacgcggugaaugcc

SEQ ID NO: 194 - RNA, 24 nt, Homo sapiens
ucccugagacccuuuaaccuguga

SEQ ID NO: 195 - RNA, 22 nt, Homo sapiens
ucccugagacccuaacuuguga

SEQ ID NO: 196 - RNA, 22 nt, Homo sapiens
acggguuaggcucuugggagcu

SEQ ID NO: 197 - RNA, 22 nt, Homo sapiens
ucacaagucaggcucuugggac

SEQ ID NO: 198 - RNA, 22 nt, Homo sapiens
ucguaccgugaguaauaaugcg

SEQ ID NO: 199 - RNA, 22 nt, Homo sapiens
ucggauccgucugagcuuggcu

SEQ ID NO: 200 - RNA, 21 nt, Homo sapiens
ucacagugaaccggucucuuu

SEQ ID NO: 201 - RNA, 21 nt, Homo sapiens
cuuuuugcggucugggcuugc

SEQ ID NO: 202 - RNA, 21 nt, Homo sapiens
acucuuucccuguugcacuac

SEQ ID NO: 203 - RNA, 22 nt, Homo sapiens
uaacagucuacagccauggucg

SEQ ID NO: 204 - RNA, 23 nt, Homo sapiens
uauggcuuuucauuccuauguga

SEQ ID NO: 205 - RNA, 22 nt, Homo sapiens
auguagggcuaaaagccauggg

SEQ ID NO: 206 - RNA, 22 nt, Homo sapiens
gcuauuucacgacaccaggguu

SEQ ID NO: 207 - RNA, 22 nt, Homo sapiens
ggagacgcggcccuguuggagu

SEQ ID NO: 208 - RNA, 21 nt, Homo sapiens
uaccacaggguagaaccacgg

SEQ ID NO: 209 - RNA, 22 nt, Homo sapiens
cagugguuuuacccuaugguag

SEQ ID NO: 210 - RNA, 22 nt, Homo sapiens
ugagaacugaauuccauaggcu

SEQ ID NO: 211 - RNA, 22 nt, Homo sapiens
ucagugcaucacagaacuuugu

SEQ ID NO: 212 - RNA, 21 nt, Homo sapiens
agggagggacgggggcugugc

SEQ ID NO: 213 - RNA, 21 nt, Homo sapiens
cuagacugaagcuccuugagg

SEQ ID NO: 214 - RNA, 21 nt, Homo sapiens
ucgaggagcucacagucuagu

SEQ ID NO: 215 - RNA, 23 nt, Homo sapiens
caaagugcuuacagugcagguag

SEQ ID NO: 216 - RNA, 23 nt, Homo sapiens
aacauucaacgcugucggugagu

SEQ ID NO: 217 - RNA, 23 nt, Homo sapiens
aacauucauugcugucggugggu

SEQ ID NO: 218 - RNA, 22 nt, Homo sapiens
aaccaucgaccguugaguggac

SEQ ID NO: 219 - RNA, 22 nt, Homo sapiens
gugaauuaccgaagggccauaa

SEQ ID NO: 220 - RNA, 22 nt, Homo sapiens
gcccaaaggugaauuuuuuggg

SEQ ID NO: 221 - RNA, 23 nt, Homo sapiens
uaaggugcaucuagugcagauag

SEQ ID NO: 222 - RNA, 22 nt, Homo sapiens
gcugcgcuuggauuucgucccc

SEQ ID NO: 223 - RNA, 22 nt, Homo sapiens
cugccaauuccauaggucacag

SEQ ID NO: 224 - RNA, 22 nt, Homo sapiens
aacuggccuacaaagucccagu

SEQ ID NO: 225 - RNA, 22 nt, Homo sapiens
aacuggcccucaaagucccgcu

SEQ ID NO: 226 - RNA, 22 nt, Homo sapiens
ccaguggggcugcuguuaucug

SEQ ID NO: 227 - RNA, 22 nt, Homo sapiens
acaguagucugcacauugguua

SEQ ID NO: 228 - RNA, 23 nt, Homo sapiens
cccaguguuuagacuaucuguuc

SEQ ID NO: 229 - RNA, 23 nt, Homo sapiens
ugugcaaaucuaugcaaaacuga

SEQ ID NO: 230 - RNA, 23 nt, Homo sapiens
ugugcaaauccaugcaaaacuga

SEQ ID NO: 231 - RNA, 22 nt, Homo sapiens
uaacacugucugguaacgaugu

SEQ ID NO: 232 - RNA, 22 nt, Homo sapiens
uaauacugccugguaaugauga

SEQ ID NO: 233 - RNA, 22 nt, Homo sapiens
caucuuacugggcagcauugga

SEQ ID NO: 234 - RNA, 23 nt, Homo sapiens
uaauacugccggguaaugaugga

SEQ ID NO: 235 - RNA, 20 nt, Homo sapiens
agagguauagggcaugggaa

SEQ ID NO: 236 - RNA, 22 nt, Homo sapiens
uggaauguaaggaagugugugg

SEQ ID NO: 237 - RNA, 21 nt, Homo sapiens
caacaccagucgaugggcugu

SEQ ID NO: 238 - RNA, 22 nt, Homo sapiens
cugugcgugugacagcggcuga

SEQ ID NO: 239 - RNA, 22 nt, Homo sapiens
uucccuuugucauccuucgccu

SEQ ID NO: 240 - RNA, 22 nt, Homo sapiens
acagcaggcacagacaggcagu

SEQ ID NO: 241 - RNA, 22 nt, Homo sapiens
gggguuccuggggaugggauuu

SEQ ID NO: 242 - RNA, 21 nt, Homo sapiens
aucacauugccagggauuacc

SEQ ID NO: 243 - RNA, 22 nt, Homo sapiens
uggcucaguucagcaggaacag

SEQ ID NO: 244 - RNA, 22 nt, Homo sapiens
uucaaguaauccaggauaggcu

SEQ ID NO: 245 - RNA, 22 nt, Homo sapiens
cacuagauugugagcuccugga

SEQ ID NO: 246 - RNA, 22 nt, Homo sapiens
aaggagcucacagucuauugag

SEQ ID NO: 247 - RNA, 21 nt, Homo sapiens
agggcccccccucaauccugu

SEQ ID NO: 248 - RNA, 24 nt, Homo sapiens
gcugguuucauauggugguuuaga

SEQ ID NO: 249 - RNA, 22 nt, Homo sapiens
uuuaacauggggguaccugcug

SEQ ID NO: 250 - RNA, 22 nt, Homo sapiens
uguaaacauccccgacuggaag

SEQ ID NO: 251 - RNA, 22 nt, Homo sapiens
ugcuaugccaacauauugccau

SEQ ID NO: 252 - RNA, 22 nt, Homo sapiens
aaaagcuggguugagagggcga

SEQ ID NO: 253 - RNA, 22 nt, Homo sapiens
cuggcccucucugcccuuccgu

SEQ ID NO: 254 - RNA, 21 nt, Homo sapiens
gccccugggccuauccuagaa

SEQ ID NO: 255 - RNA, 22 nt, Homo sapiens
cuagguauggucccagggaucc

SEQ ID NO: 256 - RNA, 23 nt, Homo sapiens
ucucacacagaaaucgcacccgu

SEQ ID NO: 257 - RNA, 21 nt, Homo sapiens
aggggugcuaucugugauuga

SEQ ID NO: 258 - RNA, 23 nt, Homo sapiens
uaggcagugucauuagcugauug

SEQ ID NO: 259 - RNA, 22 nt, Homo sapiens
uuaucagaaucuccagggguac

SEQ ID NO: 260 - RNA, 22 nt, Homo sapiens
aauugcacgguauccaucugua

SEQ ID NO: 261 - RNA, 22 nt, Homo sapiens
uaaugccccuaaaaauccuuau

SEQ ID NO: 262 - RNA, 22 nt, Homo sapiens
aauugcacuuuagcaaugguga

SEQ ID NO: 263 - RNA, 23 nt, Homo sapiens
aagugccgccaucuuuugagugu

SEQ ID NO: 264 - RNA, 23 nt, Homo sapiens
gaagugcuucgauuuuggggugu

SEQ ID NO: 265 - RNA, 22 nt, Homo sapiens
uuauaauacaaccugauaagug

SEQ ID NO: 266 - RNA, 22 nt, Homo sapiens
auauaauacaaccugcuaagug

SEQ ID NO: 267 - RNA, 21 nt, Homo sapiens
aacauagaggaaauuccacgu

SEQ ID NO: 268 - RNA, 22 nt, Homo sapiens
gaauguugcucggugaaccccu

SEQ ID NO: 269 - RNA, 22 nt, Homo sapiens
cagcagcaauucauguuuugaa

SEQ ID NO: 270 - RNA, 21 nt, Homo sapiens
caaaacgugaggcgcugcuau

SEQ ID NO: 271 - RNA, 23 nt, Homo sapiens
aaugacacgaucacucccguuga

SEQ ID NO: 272 - RNA, 22 nt, Homo sapiens
uaauacugucugguaaaaccgu

SEQ ID NO: 273 - RNA, 23 nt, Homo sapiens
ucuuggaguaggucauugggugg

SEQ ID NO: 274 - RNA, 21 nt, Homo sapiens
gcaguccaugggcauauacac

SEQ ID NO: 275 - RNA, 21 nt, Homo sapiens
ucacuccucuccucccgucuu

SEQ ID NO: 276 - RNA, 22 nt, Homo sapiens
gucauacacggcucuccucucu

SEQ ID NO: 277 - RNA, 22 nt, Homo sapiens
uccuguacugagcugccccgag

SEQ ID NO: 278 - RNA, 22 nt, Homo sapiens
aguggggaacccuuccaugagg

SEQ ID NO: 279 - RNA, 22 nt, Homo sapiens
ugaaggucuacugugugccagg

SEQ ID NO: 280 - RNA, 23 nt, Homo sapiens
uaauccuugcuaccugggugaga

SEQ ID NO: 281 - RNA, 22 nt, Homo sapiens
aaugcacccgggcaaggauucu

SEQ ID NO: 282 - RNA, 22 nt, Homo sapiens
aauccuuugucccugggugaga

SEQ ID NO: 283 - RNA, 22 nt, Homo sapiens
aaugcaccugggcaaggauuca

SEQ ID NO: 284 - RNA, 22 nt, Homo sapiens
gggagccaggaaguauugaugu

SEQ ID NO: 285 - RNA, 23 nt, Homo sapiens
uacuccagagggcgucacucaug

SEQ ID NO: 286 - RNA, 22 nt, Homo sapiens
uacugcagacguggcaaucaug

SEQ ID NO: 287 - RNA, 22 nt, Homo sapiens
aagugcugucauagcugagguc

SEQ ID NO: 288 - RNA, 23 nt, Homo sapiens
uaaauuucaccuuucugagaagg

SEQ ID NO: 289 - RNA, 22 nt, Homo sapiens
cucuagagggaagcacuuucug

SEQ ID NO: 290 - RNA, 22 nt, Homo sapiens
caaagugccucccuuuagagug

SEQ ID NO: 291 - RNA, 22 nt, Homo sapiens
aaagugcuucccuuuggacugu

SEQ ID NO: 292 - RNA, 22 nt, Homo sapiens
aaagugcuucucuuuggugggu

SEQ ID NO: 293 - RNA, 22 nt, Homo sapiens
gaaagugcuuccuuuuagaggc

SEQ ID NO: 294 - RNA, 22 nt, Homo sapiens
ccucccacacccaaggcuugca

SEQ ID NO: 295 - RNA, 22 nt, Homo sapiens
caugccuugaguguaggaccgu

SEQ ID NO: 296 - RNA, 23 nt, Homo sapiens
ucggggaucaucaugucacgaga

SEQ ID NO: 297 - RNA, 22 nt, Homo sapiens
gaaaucaagcgugggugagacc

SEQ ID NO: 298 - RNA, 21 nt, Homo sapiens
aacaggugacugguuagacaa

SEQ ID NO: 299 - RNA, 19 nt, Homo sapiens
agguugacauacguuuccc

SEQ ID NO: 300 - RNA, 20 nt, Homo sapiens
guccgcucggcgguggccca

SEQ ID NO: 301 - RNA, 22 nt, Homo sapiens
uuaugguuugccugggacugag

SEQ ID NO: 302 - RNA, 21 nt, Homo sapiens
aagccugcccggcuccucggg

SEQ ID NO: 303 - RNA, 22 nt, Homo sapiens
uggucuaggauuguuggaggag

SEQ ID NO: 304 - RNA, 23 nt, Homo sapiens
uaaaucccauggugccuucuccu

SEQ ID NO: 305 - RNA, 21 nt, Homo sapiens
ugagcuaaaugugugcuggga

SEQ ID NO: 306 - RNA, 20 nt, Homo sapiens
aggaauguuccuucuuugcc

SEQ ID NO: 307 - RNA, 22 nt, Homo sapiens
uccgagccugggucucccucuu

SEQ ID NO: 308 - RNA, 22 nt, Homo sapiens
agucauuggaggguuugagcag

SEQ ID NO: 309 - RNA, 22 nt, Homo sapiens
gacuauagaacuuucccccuca

SEQ ID NO: 310 - RNA, 21 nt, Homo sapiens
uggguuuacguugggagaacu

SEQ ID NO: 311 - RNA, 22 nt, Homo sapiens
guucucccaacguaagcccagc

SEQ ID NO: 312 - RNA, 19 nt, Homo sapiens
gugucugcuuccuguggga

SEQ ID NO: 313 - RNA, 21 nt, Homo sapiens
aggaggcagcgcucucaggac

SEQ ID NO: 314 - RNA, 25 nt, Homo sapiens
ggcggagggaaguagguccguuggu

SEQ ID NO: 315 - RNA, 23 nt, Homo sapiens
uggugcggagagggcccacagug

SEQ ID NO: 316 - RNA, 23 nt, Homo sapiens
aaggagcuuacaaucuagcuggg

SEQ ID NO: 317 - RNA, 20 nt, Homo sapiens
cggcucugggucugugggga

SEQ ID NO: 318 - RNA, 23 nt, Homo sapiens
ucugcucauaccccaugguuucu

SEQ ID NO: 319 - RNA, 28 nt, Homo sapiens
ucacaaugcugacacucaaacugcugac

SEQ ID NO: 320 - RNA, 26 nt, Homo sapiens
guuggaggaugaaaguacggagugau

SEQ ID NO: 321 - RNA, 23 nt, Homo sapiens
cugggaucuccggggucuugguu

SEQ ID NO: 322 - RNA, 22 nt, Homo sapiens
ugagaccucuggguucugagcu

SEQ ID NO: 323 - RNA, 24 nt, Homo sapiens
gauugcucugcgugcggaaucgac

SEQ ID NO: 324 - RNA, 20 nt, Homo sapiens
guagaggagauggcgcaggg

SEQ ID NO: 325 - RNA, 21 nt, Homo sapiens
uccucuucucccuccucccag

SEQ ID NO: 326 - RNA, 22 nt, Homo sapiens
uccauuacacuacccugccucu

SEQ ID NO: 327 - RNA, 21 nt, Homo sapiens
cgcgggugcuuacugacccuu

SEQ ID NO: 328 - RNA, 21 nt, Homo sapiens
uacuuggaaaggcaucaguug

SEQ ID NO: 329 - RNA, 22 nt, Homo sapiens
cacuggcuccuuucuggguaga

SEQ ID NO: 330 - RNA, 22 nt, Homo sapiens
uauugcacuugucccggccugu

SEQ ID NO: 331 - RNA, 22 nt, Homo sapiens
auccgcgcucugacucucugcc

SEQ ID NO: 332 - RNA, 21 nt, Homo sapiens
aaggcagggcccccgcucccc

SEQ ID NO: 333 - RNA, 23 nt, Homo sapiens
uuuggcacuagcacauuuuugcu

SEQ ID NO: 334 - RNA, 22 nt, Homo sapiens
cacccguagaaccgaccuugcg